(19) United States
(12) Patent Application Publication
(10) Pub. No.: US 2006/0134609 A1
(43) Pub. Date: Jun. 22, 2006

(54) Compositions and methods for determining the presence of SARS coronavirus in a sample

(76) Inventors: Jeffrey M. Linnen, Poway, CA (US); Daniel L. Kacian, San Diego, CA (US); Norman C. Nelson, San Diego, CA (US); Damon K. Getman, Poway, CA (US); Sangeetha Vijaysri, San Diego, CA (US)

Correspondence Address: GEN PROBE INCORPORATED, 10210 GENETIC CENTER DRIVE, SAN DIEGO, CA, 92121, US

(21) Appl. No.: 10/825,757

(22) Filed: Apr. 16, 2004

Related U.S. Application Data
(60) Provisional application No. 60/469,294, filed on May 9, 2003.
(60) Provisional application No. 60/465,428, filed on Apr. 25, 2003.
(60) Provisional application No. 60/464,049, filed on Apr. 17, 2003.

(51) Int. Cl. C12Q 1/70; C07H 21/04

(52) U.S. Cl. 435/5; 536/24.1

(57) ABSTRACT

The present invention relates to oligonucleotides useful for determining the presence of SARS coronavirus in a test sample. The oligonucleotides of the present invention may be incorporated into detection probes, capture probes and amplification oligonucleotides, or used in various combinations thereof.

### CROSS-REFERENCE TO RELATED APPLICATIONS

This application claims the benefit of U.S. Provisional Application No. 60/469,294, filed May 9, 2003, U.S. Provisional Application No. 60/465,428, filed Apr. 25, 2003, and U.S. Provisional Application No. 60/464,049, filed Apr. 17, 2003, the entire contents of each of these applications being hereby incorporated by reference herein.

### FIELD OF THE INVENTION

The present invention relates to compositions and methods for use in determining the presence of nucleic acid derived from a novel coronavirus associated with severe acute respiratory syndrome (SARS) as an indication of the presence of a SARS coronavirus (SARS-CoV) in a test sample.

### BACKGROUND OF THE INVENTION

A novel coronavirus has been identified that causes serious disease in humans. The disease manifests itself with a constellation of clinical findings that have been named the “severe acute respiratory syndrome” or “SARS”. The virus was first identified in China and has shown potential to spread rapidly to other countries. There is no known treatment and there has been a high fatality rate among patients who have presented with pneumonia due to the virus. The signs and symptoms of SARS are common to many diseases. At present, isolation of the patient for periods of 10 days after resolution of disease is recommended to stem the spread of the disease.

The genome of SARS-CoV was recently sequenced and initial diagnostic tests have been developed, including tests to detect antibodies to the virus and polymerase chain reaction (PCR) assays to detect viral sequences. The antibody tests are inadequate because 10-14 days or more are required for antibodies to the virus to develop to detectable levels. The PCR tests initially developed appeared to be highly specific but were sensitive in only about 50% of suspected cases. These PCR tests all amplified a sequence located in the region from about nucleotide 15000 to nucleotide 19000 in the genome.

The low sensitivity of these initial PCR tests may have several causes. For example, the PCR primers may be cross-reacting with other sequences in the samples, thereby resulting in the production of unwanted amplification products. Also, the amount of nucleic acid from SARS-CoV may be below a threshold level of detection or inhibitors in the reaction mixture may be digesting the target nucleic acid or interfering with amplification and/or detection. In addition, because SARS-CoV contains genomic RNA, these initial PCR tests may be performing an inefficient reverse transcription step prior to amplification by PCR. Thus, a need exists for a method which allows for the rapid, sensitive and specific detection of SARS-CoV nucleic acid in a test sample. And for such a method to be of clinical significance, it should be capable of distinguishing the presence of SARS-CoV from that of human coronavirus strains 229E (HCoV-229E) and OC43 (HCoV-OC43), as these latter two viruses are responsible for about 30% of mild upper respiratory tract illnesses.

### SUMMARY OF THE INVENTION

It is a principal object of the present invention to provide compositions and methods for the sensitive and specific detection of SARS-CoV derived nucleic acid in a test sample which are superior to currently available PCR methods.

In one embodiment of the present invention, a detection probe is provided for use in determining the presence of SARS-CoV in a test sample, where the probe is up to 100 bases in length and comprises a target binding portion which forms a hybrid stable for detection with a target sequence contained within the following sequence or its complement under stringent hybridization conditions:

| | |
|---|---|
| ccuuauggguugggauuaucc. | SEQ ID NO: 1 |

The probe of this embodiment does not form a hybrid stable for detection with nucleic acid derived from HCoV-OC43 or HCoV-229E under the stringent hybridization conditions.

The target binding portion of the probe is preferably substantially complementary to the target sequence or its complement. More preferably, the target binding region comprises an at least 10 contiguous base region which is perfectly complementary to an at least 10 contiguous base region of the target sequence or its complement, and more preferably comprises an at least 15 contiguous base region which is perfectly complementary to an at least 15 contiguous base region of the target sequence or its complement. In a preferred embodiment, the probe comprises a base sequence selected from the group consisting the following base sequence, its complement, and the RNA equivalents thereof:

| | |
|---|---|
| ccttatgggttgggattatcc. | SEQ ID NO:2 |

In a more preferred embodiment, the base sequence of the target binding portion is perfectly complementary to all or a portion of the base sequence of SEQ ID NO:1 or its complement, and the probe does not comprise any other base sequences which stably hybridize to nucleic acid derived from SARS-CoV under the stringent hybridization conditions. For this embodiment, the target binding portion of the probe preferably comprises an at least 10 contiguous base region which is perfectly complementary to an at least 10 contiguous base region of the target sequence or its complement, and more preferably comprises an at least 15 contiguous base region which is perfectly complementary to an at least 15 contiguous base region of the target sequence or its complement. And in a most preferred embodiment, the base sequence of the probe consists of a base sequence selected from the group consisting of SEQ ID NO:2, its complement, and the RNA equivalents thereof.

In another embodiment of the present invention, a detection probe is provided for use in determining the presence of SARS-CoV in a test sample, where the probe is up to 100 bases in length and comprises a target binding portion which forms a hybrid stable for detection with a target sequence contained within the following sequence or its complement under stringent hybridization conditions:

| | |
|---|---|
| cgugcguggauuggcuuugaugu. | SEQ ID NO:3 |

The probe of this embodiment does not form a hybrid stable for detection with nucleic acid derived from HCoV-OC43 or HCoV-229E under the stringent hybridization conditions.

The target binding region of the probe is preferably substantially complementary to the target sequence or its complement. More preferably, the target binding region comprises an at least 10 contiguous base region which is perfectly complementary to an at least 10 contiguous base region of the target sequence or its complement, and more preferably comprises an at least 15 contiguous base region which is perfectly complementary to an at least 15 contiguous base region of the target sequence or its complement.

In a preferred embodiment, the base sequence of the target binding portion is perfectly complementary to all or a portion of the base sequence of SEQ ID NO:3 or its complement, and the probe does not comprise any other base sequences which stably hybridize to nucleic acid derived from SARS-CoV under the stringent hybridization conditions. For this embodiment, the probe preferably comprises an at least 10 contiguous base region which is perfectly complementary to an at least 10 contiguous base region of the target sequence or its complement, and more preferably comprises an at least 15 contiguous base region which is perfectly complementary to an at least 15 contiguous base region of the target sequence or its complement.

In a particularly preferred embodiment, the probe comprises a base sequence selected from the group consisting the following base sequences, their complements, and the RNA equivalents thereof:

| | |
|---|---|
| cgtgcgtggattggcttt, | SEQ ID NO:4 |
| cgtgcgtggattggctttg, | SEQ ID NO:5 |
| and | |
| tgcgtggattggctttgatgt. | SEQ ID NO:6 |

In a more preferred embodiment, the base sequence of the target binding portion of the probe is contained within a base sequence selected from the group consisting of SEQ ID NO:4, SEQ ID NO:5 and SEQ ID NO:6, their complements, and the RNA equivalents thereof, and the probe does not comprise any other base sequences which stably hybridize to nucleic acid derived from SARS-CoV under the stringent hybridization conditions. The following probe sequence exemplifies a probe capable of forming a hairpin molecule through self-hybridization at its end portions (see complementary, underlined portions) under the stringent hybridization conditions, where the target binding portion of the probe is contained within the RNA equivalent of the base sequence of SEQ ID NO:4:

| | |
|---|---|
| ccgugcguggauuggcuuucacgg. | SEQ ID NO: 7 |

In an even more preferred embodiment, the base sequence of the target binding portion of the probe is selected from the group consisting of a base sequence selected from the group consisting of SEQ ID NO:4, SEQ ID NO:5 and SEQ ID NO:6, their complements, and the RNA equivalents thereof. And in a most preferred embodiment, the base sequence of the probe consists of a base sequence selected from the group consisting of SEQ ID NO:4, SEQ ID NO:5 and SEQ ID NO:6, their complements, and the RNA equivalents thereof.

The target binding portion of a detection probe may consist of DNA, RNA, a combination DNA and RNA, or it may be a nucleic acid analog (e.g., a peptide nucleic acid) or contain one or more modified nucleosides (e.g., a ribonucleoside having a 2′-O-methyl substitution to the ribofuranosyl moiety). Probes of the present invention are preferably oligonucleotides from 10 to 100 bases in length, more preferably from 15 to 50 bases in length, and most preferably from 18 to 20, 25, 30 or 35 bases in length.

Detection probes of the present invention may include one or more base sequences in addition to the base sequence of the target binding portion which do not stably bind to nucleic acid derived from SARS-CoV under stringent hybridization conditions. An additional base sequence may be comprised of any desired base sequence, so long as it does not stably bind to nucleic acid derived from SARS-CoV under stringent hybridization conditions or prevent stable hybridization of the probe to the target nucleic acid. By way of example, an additional base sequence may constitute the immobilized probe binding region of a capture probe, where the immobilized probe binding region is comprised of, for example, a 3′ poly dA (adenine) region which hybridizes under stringent conditions to a 5′ poly dT (thymine) region of a polynucleotide bound directly or indirectly to a solid support. An additional base sequence might also be a 5′ sequence recognized by an RNA polymerase or which enhances initiation or elongation by an RNA polymerase (e.g., a promoter sequence recognized by an RNA polymerase). More than one additional base sequence may be included if the target binding portion is incorporated into, for example, a self-hybridizing probe (i.e., a probe having distinct base regions capable of hybridizing to each other in the absence of target sequence under the conditions of an assay), such as a “molecular beacon” probe. Molecular beacon probes are disclosed by Tyagi et al., “Detectably Labeled Dual Conformation Oligonucleotide Probes, Assays and Kits,” U.S. Pat. No. 5,925,517, and include a target binding portion which is bounded by or overlaps with two base sequences having regions, referred to as “stems” or “arms” which are at least partially complementary to each other. A more detailed description of molecular beacon probes is provided infra in the section entitled “Useful Labeling Systems and Detectable Moieties.” An additional base sequence may be joined directly to the target binding portion or, for example, by means of a non-nucleotide linker (e.g., polyethylene glycol or an abasic region).

While not required, the detection probes preferably include a detectable label or group of interacting labels. The label may be any suitable labeling substance, including but not limited to a radioisotope, an enzyme, an enzyme cofactor, an enzyme substrate, a dye, a hapten, a chemiluminescent molecule, a fluorescent molecule, a phosphorescent molecule, an electrochemiluminescent molecule, a chromophore, a base sequence region that is unable to stably bind to the target nucleic acid under the stated conditions, and mixtures of these. In one particularly preferred embodiment, the label is an acridinium ester (AE), preferably 4-(2-succinimidyloxycarbonyl ethyl)-phenyl-10-methylacridinium-9-carboxylate fluorosulfonate (hereinafter referred to as “standard AE”). Groups of interacting labels useful with a probe pair (see, e.g., Morrison, “Competitive Homogeneous Assay,” U.S. Pat. No. 5,928,862) or a self-hybridizing probe (see, e.g., Tyagi et al., U.S. Pat. No. 5,925,517) include, but are not limited to, enzyme/substrate, enzyme/cofactor, luminescent/quencher, luminescent/adduct, dye dimers and Förrester energy transfer pairs. An interacting luminescent/quencher pair is particularly preferred, such as fluoroscein and DABCYL.

In yet another embodiment of the present invention, a method is provided for determining the presence of SARS-CoV in a test sample. In this method, any of the above-described probes is contacted with a test sample suspected of containing SARS-CoV under stringent hybridization conditions. After the probes have had sufficient time to hybridize to SARS-CoV-derived nucleic acid present in the test sample, the test sample is screened for the presence of probe:target hybrids indicative of the presence of SARS-CoV in the test sample. The SARS-CoV-derived nucleic acid may be naturally occurring SARS-CoV nucleic acid, such as genomic RNA or messenger RNA (mRNA), or it may be an amplicon thereof.

In a further embodiment of the present invention, a first oligonucleotide set is provided which comprises two or more oligonucleotides capable of amplifying a target region of nucleic acid derived from SARS-CoV under amplification conditions, where the target region is contained within the following sequence or its complement:

SEQ ID NO:8

cugugguaauuggaacaagcaaguuuuacgguggcuggcauaauauguua
aaaacuguuuacagugaugagaaacuccacaccuuauggguugggauuau
ccaaaaugugacagagccaugccuaacaugcuuaggauaauggccucucu
uguucuugcucgcaaacauaacacuugcugua.

The oligonucleotide set of this embodiment preferably includes first and second oligonucleotides, where each oligonucleotide is up to 100 bases in length, and where the first oligonucleotide of the set binds to or extends through a target sequence contained within the following sequence or its complement under amplification conditions:

SEQ ID NO:9

uauccaaaaugugacagagccaugccuaacaugcuuaggauaauggccuc
ucuuguucuug cucgcaaacauaacacuugcugua.

The second oligonucleotide of the set binds to or extends through a target sequence contained within the following sequence or its complement under amplification conditions:

SEQ ID NO: 10

cugugguaauuggaacaagcaaguuuuacgguggcugg.

The target sequence of the first oligonucleotide is preferably selected from the following group of sequences and their complements:

| | |
|---|---|
| uauccaaaaugugacagagccaugcc, | SEQ ID NO: 11 |
| auccaaaaugugacagagccaugc, | SEQ ID NO: 12 |
| ccaaaaugugacagagccaugcc, | SEQ ID NO: 13 |
| aaaugugacagagccaugccuaa, | SEQ ID NO: 14 |
| ugugacagagccaugccuaacaugcu, | SEQ ID NO: 15 |
| gugacagagccaugccuaacaugcu, | SEQ ID NO: 16 |
| augccuaacaugcuuaggauaau, | SEQ ID NO: 17 |
| augcuuaggauaauggccucu, | SEQ ID NO: 18 |
| and | |
| gcucgcaaacauaacacuugcugua. | SEQ ID NO: 19 |

More particularly, the first oligonucleotide preferably has a base sequence which comprises or substantially corresponds to a base sequence selected from the group consisting of SEQ ID NO:11, SEQ ID NO:12, SEQ ID NO:13, SEQ ID NO:14, SEQ ID NO:15, SEQ ID NO:16, SEQ ID NO:17, SEQ ID NO:18 and SEQ ID NO:19, their complements, and the DNA equivalents thereof. Even more particularly, the base sequence of the first oligonucleotide is preferably a base sequence consisting of or contained within a base sequence selected from the group consisting of SEQ ID NO:11, SEQ ID NO:12, SEQ ID NO:13, SEQ ID NO:14, SEQ ID NO:15, SEQ ID NO:16, SEQ ID NO:17, SEQ ID NO:18 and SEQ ID NO:19, their complements, the DNA equivalents thereof, and any of the foregoing in combination with a 5′ sequence which is recognized by an RNA polymerase or which enhances initiation or elongation by RNA polymerase (e.g., a promoter sequence for an RNA polymerase).

The target sequence of the second oligonucleotide is preferably selected from the following group of sequences and their complements:

| | |
|---|---|
| cugugguaauuggaacaagcaaguu, | SEQ ID NO:20 |
| gaacaagcaaguuuuacgg, | SEQ ID NO:21 |
| and | |
| aagcaaguuuuacgguggcugg. | SEQ ID NO:22 |

More particularly, the second oligonucleotide preferably has a base sequence which comprises or substantially corresponds to a base sequence selected from the group consisting of SEQ ID NO:20, SEQ ID NO:21 and SEQ ID NO:22, their complements, and the DNA equivalents thereof. Even more particularly, the base sequence of the second oligonucleotide is preferably a base sequence consisting of or contained within a base sequence selected from the group consisting of SEQ ID NO:20, SEQ ID NO:21 and SEQ ID NO:22, their complements, the DNA equivalents thereof, and any of the foregoing in combination with a 5′ sequence which is recognized by an RNA polymerase or which enhances initiation or elongation by RNA polymerase (e.g., a promoter sequence for an RNA polymerase).

In still another embodiment of the present invention, a second oligonucleotide set is provided which comprises two or more oligonucleotides capable of amplifying a target region of nucleic acid derived from SARS-CoV under amplification conditions, where the target region is contained within the following sequence or its complement:

SEQ ID NO:23

caagucaaugguuacccuaauauguuuaucacccgcgaagaagcuauucg
ucacguucgugcguggauuggcuuugauguagagggcugucaugcaacua
gagaugcugugg.

The oligonucleotide set of this embodiment preferably includes first and second oligonucleotides, where each oligonucleotide is up to 100 bases in length, and where the first oligonucleotide of the set binds to or extends through a target sequence contained within the following sequence or its complement under amplification conditions:

| | |
|---|---|
| gagggcugucaugcaacuagagaugcugugg. | SEQ ID NO:24 |

The second oligonucleotide of the set binds to or extends through a target sequence contained within the following sequence or its complement under amplification conditions:

SEQ ID NO:25

caagucaaugguuacccuaauauguuuaucacccgcgaagaagcu.

The target sequence of the first oligonucleotide is preferably selected from the following group of sequences and their complements:

| | |
|---|---|
| gagggcugucaugcaacuaga, | SEQ ID NO:26 |
| and | |
| caugcaacuagagaugcugugg. | SEQ ID NO:27 |

More particularly, the first oligonucleotide preferably has a base sequence which comprises or substantially corresponds to a base sequence selected from the group consisting of SEQ ID NO:26 and SEQ ID NO:27, their complements, and the DNA equivalents thereof. Even more particularly, the base sequence of the first oligonucleotide is preferably a base sequence consisting of or contained within a base sequence selected from the group consisting of SEQ ID NO:26 and SEQ ID NO:27, their complements, the DNA equivalents thereof, and any of the foregoing in combination with a 5′ sequence which is recognized by an RNA polymerase or which enhances initiation or elongation by RNA polymerase (e.g., a promoter sequence for an RNA polymerase).

The target sequence of the second oligonucleotide is preferably selected from the following group of sequences and their complements:

| | |
|---|---|
| caagucaaugguuacccuaauaug, | SEQ ID NO:28 |
| gucaaugguuacccuaauauguu, | SEQ ID NO:29 |
| caaugguuacccuaauauguuuau, | SEQ ID NO:30 |
| uuacccuaauauguuuaucacc, | SEQ ID NO:31 |
| cuaauauguuuaucacccgcg, | SEQ ID NO:32 |
| and | |
| uuaucacccgcgaagaagcu. | SEQ ID NO:33 |

More particularly, the second oligonucleotide preferably has a base sequence which comprises or substantially corresponds to a base sequence selected from the group consisting of SEQ ID NO:28, SEQ ID NO:29, SEQ ID NO:30, SEQ ID NO:31, SEQ ID NO:32 and SEQ ID NO:33, their complements, and the DNA equivalents thereof. Even more particularly, the base sequence of the second oligonucleotide is preferably a base sequence consisting of or contained within a base sequence selected from the group consisting of SEQ ID NO:28, SEQ ID NO:29, SEQ ID NO:30, SEQ ID NO:31, SEQ ID NO:32 and SEQ ID NO:33, their complements, the DNA equivalents thereof, and any of the foregoing in combination with a 5′ sequence which is recognized by an RNA polymerase or which enhances initiation or elongation by RNA polymerase (e.g., a promoter sequence for an RNA polymerase).

While amplification oligonucleotides of the present invention may vary in length, the target binding portions of preferred amplification oligonucleotides are from 18 to 40 bases in length with a predicted Tₘ to target above 42° C., preferably at least about 50° C. As indicated above, amplification oligonucleotides of the present invention may additionally include a promoter sequence recognized by an RNA polymerase. Preferred are promoter sequences recognized by a T7, T3 or SP6 RNA polymerase. Particularly preferred is the following T7 RNA polymerase promoter sequence:

| | |
|---|---|
| aatttaatacgactcactatagggaga. | SEQ ID NO:34 |

Each of the first and second oligonucleotide sets may further comprise a third oligonucleotide for use in determining the presence of a target sequence derived from the target regions of SARS-CoV RNA. The third oligonucleotide of this embodiment is up to 100 bases in length and comprises a target binding portion which forms a hybrid stable for detection with the target sequence under stringent hybridization conditions. The third oligonucleotide does not form a hybrid stable for detection with nucleic acid derived from HCoV-OC43 or HCoV-229E under the stringent hybridization conditions. The third oligonucleotide may be any of the detection probes described supra having a target binding portion which is complementary to the sequence of SEQ ID NO:1 or its complement when included in the first oligonucleotide set and to the sequence of SEQ ID NO:3 or its complement when included in the second oligonucleotide set.

In lieu of or in addition to the third oligonucleotide described above, the first and second oligonucleotide sets may further comprise a fourth oligonucleotide for use in isolating and purifying a target nucleic acid containing the target region of SARS-CoV RNA. The fourth oligonucleotide of this embodiment is up to 100 bases in length and comprises a target binding portion that is complementary to a target sequence selected from the group consisting of:

| | |
|---|---|
| agacaguuucaaaucagaaauuauu, | SEQ ID NO:35 |
| auauguuaaaccagguggaacau, | SEQ ID NO:36 |
| and | |
| gguguuaacuuagucagcuguaccgacugg. | SEQ ID NO:37 |

The fourth oligonucleotide stably hybridizes to the target sequence under assay conditions. The base sequence of the target binding portion of the fourth oligonucleotide preferably consists of or is contained within the complements of SEQ ID NO:35, SEQ ID NO:36 and SEQ ID NO:37, and the DNA equivalents thereof. The fourth oligonucleotide includes a region or molecule permitting the fourth oligonucleotide to be bound directly or indirectly to a solid support by such means as complementary base pairing or a ligand/ligate interaction (e.g., avidin/biotin). Capture probes according to the present invention may be provided independent of the above-described oligonucleotide sets.

In another embodiment of the present invention, a method is provided for amplifying a target region of nucleic acid derived from SARS-CoV. In this method, any of the above-described oligonucleotide sets comprising first and second oligonucleotides is contacted with a test sample suspected of containing SARS-CoV. The test sample is exposed to amplification conditions and the target region, if present in the test sample, is amplified. To determine whether SARS-CoV is present in the test sample, a third oligonucleotide, as described above, which is capable of distinguishing between SARS-CoV-derived nucleic acid and nucleic acid derived from HCoV-OC43 and HCoV-229E is provided to the test sample. The third oligonucleotide may be provided to the test sample prior to, during and/or after exposure of the test sample to amplification conditions. To enhance sensitivity of the detection method, a fourth oligonucleotide may be provided to the test sample prior to contacting the test sample with the amplification oligonucleotides in order to isolate and purify the SARS-CoV-derived nucleic acid, thereby removing at least a portion of the non-target nucleic acids and inhibitors of nucleic acid that may be present in the test sample. The fourth oligonucleotide may be used in a method of isolating and purifying a target nucleic acid that does not require any of the other members of the oligonucleotide set described above.

In further embodiment of the present invention, a method is provided for determining the presence of SARS-CoV in a test sample which includes contacting a test sample with a detection probe up to 100 bases in length. In this method, any of the above-described probes is contacted with a test sample suspected of containing SARS-CoV under stringent hybridization conditions. After the probes have had sufficient time to hybridize to SARS-CoV-derived nucleic acid present in the test sample, the test sample is screened for the presence of probe:target hybrids indicative of the presence of SARS-CoV in the test sample. The SARS-CoV-derived nucleic acid may be naturally occurring SARS-CoV nucleic acid, such as genomic RNA or messenger RNA (mRNA), or it may be an amplicon thereof.

In another embodiment of the present invention, a method is provided in which multiple regions of the SARS-CoV genome are targeted for detection. In a particularly preferred embodiment, one or more of the regions selected for detection are also present in subgenomic mRNAs of the SARS-CoV, thereby further enhancing the sensitivity of the method. Targeting multiple regions of the SARS-CoV genome makes the method of the present invention less sensitive to mutations in the SARS-CoV genome and, therefore, less likely to give a false negative result if one or some of the targeted regions contain a mutation. This feature of the present invention is especially important in the case of nidoviruses, which include both coronaviruses and arteriviruses, as nidoviruses are known to have high mutation rates. Targeting multiple target sequences in the SARS-CoV genome also minimizes reductions in sensitivity that may result when viral RNA is present in a sample in low copy number, when viral RNA is lost due to degradation, adsorption onto surfaces, dilution or other causes related to specimen collection, transport, storage or sample processing. The regions targeted for detection are preferably contained within SARS-CoV amplified sequences.

Thus, in a preferred embodiment of the present invention, a method is provided in which a 5′ leader sequence or a shared 3′ terminal sequence of SARS-CoV RNA (“3′ co-terminal sequence”) is targeted for amplification and/or detection. As used herein, the term “SARS-CoV RNA” refers to the full-length plus strand genomic RNA and the set of subgenomic mRNAs generated during the life cycle of the virus when it infects a cell. The genome of SARS-CoV is a capped and polyadenylated plus strand RNA, portions of which are translated when the virus infects its host cell to produce an RNA-directed RNA polymerase (replicase) that is specific for SARS-CoV RNA. The replicase makes a complete negative strand copy of SARS-CoV genomic RNA, as well as a series of subgenomic mRNAS. Each of the subgenomic mRNAs begins with a 5′ leader sequence and ends with the same 3′ co-terminal region found in the full-length plus strand genomic RNA. Sequences contained within or derived from a 5′ leader sequence or a sequence of the 3′ co-terminal region may be detected directly or following an amplification step.

In one embodiment of this method, a test sample is contacted with a detection probe up to 100 bases in length and comprising a target binding portion which forms a hybrid stable for detection with a target sequence contained within a SARS-CoV 5′ leader sequence or its complement under stringent hybridization conditions, where the probe does not form a hybrid stable for detection with nucleic acid derived from HCoV-OC43 or HCoV-229E under the stringent hybridization conditions. The target sequence preferably comprises the core sequence of a transcription regulating sequence or its complement. The targeted core sequence preferably comprises at least 5 contiguous nucleotides of the following sequence:

| | |
|---|---|
| uaaaacgaac | SEQ ID NO:38 |

More preferably, the targeted core sequence consists of the sequence of SEQ ID NO:38 or its complement.

In a further embodiment of this method, the target sequence is produced in a method of amplification in which a test sample suspected of containing SARS-CoV is contacted with a pair of amplification oligonucleotides under amplification conditions, where each member of the pair of amplification oligonucleotides binds to or extends through at least a portion of the targeted 5′ leader sequence or its complement under the amplification conditions. The target binding portion of each member of the pair of amplification oligonucleotides preferably binds to a target region fully contained within the targeted 5′ leader sequence or its complement under the amplification conditions, where the amplification oligonucleotides do not contain any other base sequences which stably hybridize to nucleic acid derived from SARS-CoV under the amplification conditions. In an alternative method, the target binding portion of a first member of the pair of amplification oligonucleotides binds to a target region fully contained within the targeted 5′ leader sequence or its complement under the amplification conditions, and the target binding portion of the second member of the pair of amplification oligonucleotides binds to a target region fully contained within a 3′ co-terminal sequence present in SARS-CoV RNA or its complement under the amplification conditions, where the amplification oligonucleotides do not contain any other base sequences which stably hybridize to nucleic acid derived from SARS-CoV under the amplification conditions. It is preferred that at least one of the amplification oligonucleotides of this method binds to the core sequence of a transcription regulating sequence within the targeted 5′ leader sequence or its complement under the amplification conditions. The core sequence preferably consists of at least 5 contiguous nucleotides of SEQ ID NO:38 or its complement, and more preferably consists of the sequence of SEQ ID NO:38 or its complement.

In another embodiment of this method, a test sample is contacted with a detection probe up to 100 bases in length and comprising a target binding portion which forms a hybrid stable for detection with a target sequence contained within a 3′ co-terminal sequence or its complement under stringent hybridization conditions, where the probe does not form a hybrid stable for detection with nucleic acid derived from HCoV-OC43 or HCoV-229E under the stringent hybridization conditions.

In still another embodiment of this method, the target sequence is produced in a method of amplification in which a test sample suspected of containing SARS-CoV is contacted with a pair of amplification oligonucleotides under amplification conditions, where each member of the pair of amplification oligonucleotides comprises a target binding portion which binds to or extends through at least a portion of a 3′ co-terminal sequence under the amplification conditions. The target binding portion of each member of the pair of amplification oligonucleotides preferably binds to a target region fully contained within the targeted 3′ co-terminal sequence or its complement under the amplification conditions, where the amplification oligonucleotides do not contain any other base sequences which stably hybridize to nucleic acid derived from SARS-CoV under the amplification conditions.

In yet another embodiment of the present invention, the detection methods of the present invention are included in a panel test including means for determining the presence of other contributing or secondary agents that may be associated with SARS. Included in the same or an alternative panel test would be means for determining the presence of other organisms or viruses which present with the same signs or symptoms associated with SARS. Such organisms and viruses include adenoviruses, Legionella, Streptococcus pneumoniae, Chlamydia pneumoniae, Mycoplasma pneumoniae and other human respiratory coronaviruses.

The detection probes of the present invention are designed to have specificity for the SARS-CoV-derived sequences they target in a test sample, but may target regions of homology with sequences of other organisms or viruses that would not be expected to be present in the test sample (e.g., probes may target a region of the SARS-CoV genome homologous with a Zebrafish DNA sequence). Capture probes and amplification oligonucleotides of the present invention are preferably designed to have specificity for their target sequences, although this is not a requirement of the present invention.

While it is preferred that amplification products generated using the methods of the present invention are detected using detection probes specific for sequences contained within the amplification products, alternative methods are known in the art which may be employed to detect and identify the amplified sequences. These include, for example, nucleic acid sequencing, restriction fragment mapping, size separation using gel electrophoresis and high pressure liquid chromatography and mass spectroscopy.

Other features and advantages of the invention will be apparent from the following description of the preferred embodiments thereof and from the claims.

### BRIEF DESCRIPTION OF THE DRAWINGS

FIG. 1 is a graphical representation of the results of a SARS-CoV real-time amplification assay, showing 100 to 1000 copy sensitivity.

FIG. 2 is a bar chart showing 100% reactivity of a SARS-CoV amplification assay with end-point detection at 80 copies per mL.

### DETAILED DESCRIPTION OF THE PREFERRED EMBODIMENTS

Disclosed herein are methods for the selective and sensitive detection of nucleic acid derived from SARS-CoV present in a test sample, such as a nasopharyngeal swab. The methods of the present invention can be used, for example, to aid in the diagnosis of SARS or to monitor the therapeutic treatment of a SARS-CoV-infected individual.

To identify candidate oligonucleotides for use in detecting the presence of nucleic acid derived from SARS-CoV, NCBI BLAST searches were performed comparing a published viral genome of SARS-CoV (GenBank accession no. NC_{—}004718) with a database including all GenBank, EMBL, DDBJ and PDB sequences, excluding EST, STS, GSS and phase 0, 1 and 2 HTGS sequences for regions of homology with sequences derived from organisms or viruses other than SARS-CoV. Oligonucleotide sequences of particularly preferred detection probes contemplated by the present invention are intended to target regions of non-homology, but may also be designed to target SARS-CoV RNA target regions sharing sequence identity with organisms or viruses which would not be expected to be present in a sample or which would not be amplified in an amplification procedure.

In a particularly preferred embodiment, at least some of the regions targeted by oligonucleotides of the present invention include a 5′ leader sequence, preferably the 5′ leader sequence of a subgenomic mRNA derived from the 5′ end of SARS-CoV genomic RNA, and/or a 3′ terminal sequence shared by all SARS-CoV RNAs. The 5′ leader sequences of subgenomic mRNAs are derived from the region 5′ of the 5′ most gene of the SARS-CoV genome. That gene for SARS-CoV is believed to begin at about position 250 of the viral genome. The 3′ terminal region encompasses the last gene at the 3′ end of SARS-CoV RNA.

Those skilled in the art will appreciate that capture probes or amplification oligonucleotides according to the present invention do not need to be specific for nucleic acid derived from SARS-CoV if a particular application is designed to tolerate a degree of non-specific capture or amplification. Also, oligonucleotides of the present invention may serve multiple functions. For example, the target binding regions of capture probes according to the present invention could serve as detection probes, the detection probes according to the present invention could be used as amplification oligonucleotides or helper oligonucleotides, the amplification oligonucleotides could be used as detection probes or helper oligonucleotides, and the helper oligonucleotide could be used as detection probes or amplification oligonucleotides in alternative detection assays.

### A. DEFINITIONS

The following terms have the indicated meanings in the specification unless expressly indicated to have a different meaning.

By “sample” or “test sample” is meant any tissue or polynucleotide-containing material obtained from a human, animal or environmental sample. Test samples in accordance with the invention include, but are not limited to, throat or nasopharyngeal swabs or aspirates, bronchial-alveolar lavages, blood, stool and possibly sweat. A test sample may be treated to disrupt tissue or cell structure, thereby releasing intracellular components into a solution which may contain enzymes, buffers, salts, detergents and the like. Certain types of test samples will require pre-treatment, such as sputum, which can be liquified with a disulfide bond reducing agent (e.g., dithiothreitol) in combination with a DNA digestion agent (e.g., DNase), as disclosed by Kacian, “Techniques for Preparing Specimens for Bacterial Assays,” U.S. Pat. No. 5,364,763, the contents of which are hereby incorporated by reference herein. In the claims, the terms “sample” and “test sample” may refer to specimen in its raw form or to any stage of processing to release, isolate and purify nucleic acid derived from target viruses in the specimen. Thus, within a method of use claim, each reference to a “sample” or “test sample” may refer to a substance suspected of containing nucleic acid derived from the target virus at different stages of processing and is not limited to the initial form of the substance in the claim.

By “polynucleotide” is meant RNA and/or DNA, and analogs thereof that do not prevent hybridization of the polynucleotide with a second molecule having a complementary sequence.

By “detectable label” is meant a chemical species that can be detected or can lead to a detectable response. Detectable labels in accordance with the invention can be linked to polynucleotide probes either directly or indirectly, and include radioisotopes, enzymes, haptens, chromophores such as dyes or particles that impart a detectable color (e.g., latex beads or metal particles), luminescent compounds (e.g., bioluminescent, phosphorescent or chemiluminescent moieties) and fluorescent compounds.

By “interacting label pair” is meant a pair of chemical species associated with a probe that interact to emit detectably different signals, depending on whether the probe is or is not bound to a target sequence. The chemical species comprising the interacting label pair can be the same or different. Interacting label pairs include luminescent/quencher pairs, luminescent/adduct pairs, Förrester energy transfer pairs and dye dimers.

By “homogeneous detectable label” is meant a label that can be detected in a homogeneous fashion by determining whether the label is on a probe hybridized to a target sequence. That is, homogeneous detectable labels can be detected without physically removing hybridized from unhybridized forms of the label or labeled probe. These labels have been described in detail by Arnold et al., “Homogenous Protection Assay,” U.S. Pat. No. 5,283,174; Woodhead et al., “Detecting or Quantifying Multiple Analytes Using Labelling Techniques,” U.S. Pat. No. 5,656,207; and Nelson et al., “Compositions and Methods for the Simultaneous Detection and Quantification of Multiple Specific Nucleic Acid Sequences,” U.S. Pat. No. 5,658,737, each of which references is hereby incorporated by reference herein. Preferred labels for use in homogenous assays include chemiluminescent compounds. Preferred chemiluminescent labels are acridinium ester (“AE”) compounds, such as standard AE or derivatives thereof (e.g., naphthyl-AE, ortho-AE, 1- or 3-methyl-AE, 2,7-dimethyl-AE, 4,5-dimethyl-AE, ortho-dibromo-AE, ortho-dimethyl-AE, meta-dimethyl-AE, ortho-methoxy-AE, ortho-methoxy(cinnamyl)-AE, ortho-methyl-AE, ortho-fluoro-AE, 1- or 3-methyl-ortho-fluoro-AE, 1- or 3-methyl-meta-difluoro-AE, and 2-methyl-AE).

By “amplification” is meant an in vitro procedure for obtaining multiple copies of a target nucleic acid sequence, its perfect complement or fragments thereof. Copies of the target nucleic acid sequence may be DNA, RNA or both DNA and RNA.

By “amplification conditions” is meant conditions permitting nucleic acid amplification. While the Examples section infra provides preferred amplification conditions for amplifying target nucleic acid sequences derived from SARS-CoV using amplification oligonucleotides of the present invention in a transcription-based amplification method, other acceptable amplification conditions could be easily ascertained by someone having ordinary skill in the art depending on the particular method of amplification employed.

By “target nucleic acid” or “target” is meant a nucleic acid containing a target nucleic acid sequence.

By “target nucleic acid sequence” or “target sequence” or “target region” is meant a specific deoxyribonucleotide or ribonucleotide sequence comprising all or part of the nucleotide sequence of a single-stranded nucleic acid molecule, and the deoxyribonucleotide or ribonucleotide sequence perfectly complementary thereto.

By “transcription-based amplification” is meant any type of nucleic acid amplification that uses an RNA polymerase to produce multiple RNA transcripts from a nucleic acid template. Transcription-based amplification methods generally employ an RNA polymerase, a DNA polymerase, deoxyribonucleoside triphosphates, ribonucleoside triphosphates, and a template-complementary oligonucleotide containing a promoter sequence recognized by an RNA polymerase. Examples of transcription-based amplification methods include self-sustained sequence replication (3SR), transcription-mediated amplification (TMA) and nucleic acid sequence-based amplification (NASBA). See, e.g., Fahy et al., “Self-sustained Sequence Replication (3SR): An Isothermal Transcription-Based Amplification System Alternative to PCR,” PCR Methods and Applications, 1:25-33 (1991); Kacian et al., “Nucleic Acid Sequence Amplification Methods,” U.S. Pat. No. 5,399,491; Kacian et al., “Nucleic Acid Sequence Amplification Method, Composition and Kit,” U.S. Pat. No. 5,554,516; McDonough et al., “Method of Amplifying Nucleic Acids Using Promoter-Containing Primer Sequence,” Malek et al., “Enhanced Nucleic Acid Amplification Process,” U.S. Pat. No. 5,130,238; Davey et al., “Nucleic Acid Amplification Process,” U.S. Pat. No. 5,554,517; and Burg et al., “Selective Amplification of Target Polynucleotide Sequences,” U.S. Pat. No. 5,437,990. Each of the foregoing references is hereby incorporated by reference herein. The methods of Kacian et al. are preferred for conducting nucleic acid amplification procedures of the type disclosed herein.

By “oligonucleotide” or “oligomer” is meant a polymeric chain of at least two, generally between about five and about 100, chemical subunits, each subunit comprising a nucleotide base moiety, a sugar moiety, and a linking moiety that joins the subunits in a linear spatial configuration. Common nucleotide base moieties are guanine (G), adenine (A), cytosine (C), thymine (T) and uracil (U), although other rare or modified nucleotide bases able to hydrogen bond are well known to those skilled in the art. Oligonucleotides may optionally include analogs of any of the sugar moieties, the base moieties, and the backbone constituents. Preferred oligonucleotides of the present invention range in size from about 10 to about 100 residues. Oligonucleotides may be purified from naturally occurring sources, but preferably are synthesized using any of a variety of well-known enzymatic or chemical methods.

By “detection probe” or “probe” is meant a molecule comprising an oligonucleotide that hybridizes specifically to a target sequence in a nucleic acid, preferably in an amplified nucleic acid, under conditions that promote hybridization, to form a hybrid stable for detection. A probe optionally may contain a detectable moiety which either may be attached to the end(s) of the probe or may be internal. (The detectable moiety may be joined to the probe after hybridization with a target sequence as, for example, in the case of a biotinylated nucleotide.) The nucleotides of the probe which combine with the target polynucleotide need not be strictly contiguous, as may be the case with a detectable moiety internal to the sequence of the probe. Detection may either be direct (i.e., resulting from a probe hybridizing directly to the target sequence or amplified nucleic acid) or indirect (i.e., resulting from a probe hybridizing to an intermediate molecular structure that links the probe to the target sequence or amplified nucleic acid). The “target” of a probe generally refers to a sequence contained within an amplified nucleic acid sequence which hybridizes specifically to at least a portion of a probe oligonucleotide using standard hydrogen bonding (i.e., base pairing). A probe may comprise target-specific sequences and optionally other sequences that are non-complementary to the target sequence that is to be detected. These non-complementary sequences may comprise a promoter sequence, a restriction endonuclease recognition site, or sequences that contribute to three-dimensional conformation of the probe (e.g., as described in Tyagi et al., U.S. Pat. No. 5,925,517). Sequences that are “sufficiently complementary” allow stable hybridization of a probe oligonucleotide to a target sequence that is not completely complementary to the probe's target-specific sequence.

By “target binding portion” is meant a base region of an oligonucleotide which is capable of forming a stable hybrid with a target sequence under the specified conditions of use. In the case of a detection probe, the target binding portion is a base region that allows the detection probe to form a hybrid stable for detection with the target nucleic acid under stringent hybridization conditions. In the case of an amplification oligonucleotide, the target binding portion is a base region contained within a primer (i.e., primers and promoter-primers) or the template binding portion of an amplification oligonucleotide that does not have a priming function (e.g., splice template having a RNA polymerase promoter sequence which is modified at its 3′ end to prevent extension therefrom) that allows the amplification oligonucleotide to stably hybridize to the target nucleic acid under amplification conditions. And in the case of capture probes, the target binding portion is a base region that allows the capture probe to stably hybridize to the target nucleic acid under stringent conditions.

By “complement” is meant, unless otherwise indicated, a sequence which is the perfect complement of the referenced sequence (i.e., the “complement” is of the same length as and the exact complement of the referenced sequence).

By “stably,” “stable” or “stable for detection” is meant that the temperature of a reaction mixture is at least 2° C. below the melting temperature of a nucleic acid duplex. The temperature of the reaction mixture is more preferably at least 5° C. below the melting temperature of the nucleic acid duplex, and even more preferably at least 10° C. below the melting temperature of the reaction mixture.

By “helper probe” or “helper oligonucleotide” is meant an oligonucleotide designed to hybridize to a target nucleic acid at a different locus than that of a detection probe, thereby either increasing the rate of hybridization of the probe to the target nucleic acid, increasing the melting temperature (Tₘ) of the probe:target hybrid, or both.

By “amplification oligonucleotide” is meant an oligonucleotide that hybridizes to a target nucleic acid, or its perfect complement, and participates in a nucleic acid amplification reaction. Amplification oligonucleotides are generally amplification primers, but include any oligonucleotide which participates in a nucleic acid amplification reaction (see, e.g., Marshall et al., “Amplification of RNA Sequences Using the Ligase Chain Reaction,” U.S. Pat. No. 5,686,272; and Kacian et al, U.S. Pat. No. 5,399,491 (e.g., splice templates)).

By “amplification primer” or “primer” is meant an oligonucleotide capable of hybridizing to a target nucleic acid and acting as a primer and/or a promoter template (e.g., for synthesis of a complementary strand, thereby forming a functional promoter sequence) for the initiation of nucleic acid synthesis. If the amplification primer is designed to initiate RNA synthesis, the primer may contain a base sequence which is non-complementary to the target sequence but which is recognized by an RNA polymerase, such as a T7, T3 or SP6 RNA polymerase. An amplification primer may contain a 3′ terminus which is modified to prevent or lessen the rate or amount of primer extension. (McDonough et al., “Methods for Amplifying Nucleic Acids Using Promoter-Containing Primer Sequences,” U.S. Pat. No. 5,766,849, disclose primers and promoter-primers having modified or blocked 3′-ends.) While the amplification primers of the present invention may be chemically synthesized or derived from a vector, they are not naturally-occurring nucleic acid molecules.

By “substantially homologous,” “substantially corresponding” or “substantially corresponds” is meant that the subject oligonucleotide has a base sequence containing an at least 10 contiguous base region that is at least 70% homologous, preferably at least 80% homologous, more preferably at least 90% homologous, and most preferably 100% homologous to an at least 10 contiguous base region present in a reference base sequence (excluding RNA and DNA equivalents). Those skilled in the art will readily appreciate modifications that could be made to the hybridization assay conditions at various percentages of homology to permit hybridization of the oligonucleotide to the target sequence while preventing unacceptable levels of non-specific hybridization. The degree of similarity is determined by comparing the order of nucleobases making up the two sequences and does not take into consideration other structural differences which may exist between the two sequences, provided the structural differences do not prevent hydrogen bonding with complementary bases. The degree of homology between two sequences can also be expressed in terms of the number of base mismatches present in each set of at least 10 contiguous bases being compared, which may range from 0-2 base differences.

By “substantially complementary” is meant that the subject oligonucleotide has a base sequence containing an at least 10 contiguous base region that is at least 70% complementary, preferably at least 80% complementary, more preferably at least 90% complementary, and most preferably 100% complementary to an at least 10 contiguous base region present in a target nucleic acid sequence (excluding RNA and DNA equivalents). (Those skilled in the art will readily appreciate modifications that could be made to the hybridization assay conditions at various percentages of complementarity to permit hybridization of the oligonucleotide to the target sequence while preventing unacceptable levels of non-specific hybridization.) The degree of complementarity is determined by comparing the order of nucleobases making up the two sequences and does not take into consideration other structural differences which may exist between the two sequences, provided the structural differences do not prevent hydrogen bonding with complementary bases. The degree of complementarity between two sequences can also be expressed in terms of the number of base mismatches present in each set of at least 10 contiguous bases being compared, which may range from 0-2 base mismatches.

By “sufficiently complementary” is meant a contiguous nucleic acid base sequence that is capable of hybridizing to another base sequence by hydrogen bonding between a series of complementary bases. Complementary base sequences may be complementary at each position in the base sequence of an oligonucleotide using standard base pairing (e.g., G:C, A:T or A:U pairing) or may contain one or more residues that are not complementary using standard hydrogen bonding (including abasic “nucleotides”), but in which the entire complementary base sequence is capable of specifically hybridizing with another base sequence under appropriate hybridization conditions. Contiguous bases are preferably at least about 80%, more preferably at least about 90%, and most preferably about 100% complementary to a sequence to which an oligonucleotide is intended to specifically hybridize. Appropriate hybridization conditions are well known to those skilled in the art, can be predicted readily based on base sequence composition, or can be determined empirically by using routine testing (See, e.g., J. SAMBROOK ET AL., MOLECULAR CLONING: A LABORATORY MANUAL §§1.90-1.91, 7.37-7.57, 9.47-9.51, 11.12-11.13 and 11.47-11.57 (2d ed. 1989).

By “preferentially hybridize” is meant that under stringent hybridization assay conditions, detection probes of the present invention can hybridize to their target nucleic acids to form stable probe:target hybrids indicating the presence of the targeted virus (“detectable hybrids”), and there is not formed a sufficient number of stable probe:non-target hybrids to indicate the presence of non-targeted virus or organism (“non-detectable hybrids”). Thus, the probe hybridizes to target nucleic acid to a sufficiently greater extent than to non-target nucleic acid to enable one having ordinary skill in the art to accurately detect the presence (or absence) of nucleic acid derived from SARS-CoV and to distinguish its presence from other viruses or organisms that may be present in a test sample. In general, reducing the degree of complementarity between an oligonucleotide sequence and its target sequence will decrease the degree or rate of hybridization of the oligonucleotide to its target region. However, the inclusion of one or more non-complementary bases may facilitate the ability of an oligonucleotide to discriminate against non-target organisms.

Preferential hybridization can be measured using a variety of techniques known in the art, including, but not limited to those based on light emission, mass changes, changes in conductivity or turbidity. A number of detection means are described herein, and one in particular is used in the examples below. Preferably, there is at least a 10-fold difference between target and non-target hybridization signals in a test sample, more preferably at least a 100-fold difference, and most preferably at least a 500-fold difference. Preferably, non-target hybridization signals in a test sample are no more than the background signal level.

By “stringent hybridization conditions” or “stringent conditions” is meant conditions permitting a detection probe to preferentially hybridize to a target nucleic acid over nucleic acid derived from a non-target organism or virus, such as HCoV-OC43 or HCoV-229E. Stringent hybridization conditions may vary depending upon factors including the GC content and length of the probe, the degree of similarity between the probe sequence and sequences of non-target sequences which may be present in the test sample, and the target sequence. Hybridization conditions include the temperature and the composition of the hybridization reagents or solutions. While the Examples section infra provides preferred hybridization conditions for detecting target nucleic acid derived SARS-CoV using the probes of the present invention, other stringent hybridization conditions could be easily ascertained by someone having ordinary skill in the art.

By “assay conditions” is meant conditions permitting stable hybridization of an oligonucleotide (e.g., capture probe) to a target nucleic acid. Assay conditions do not require preferential hybridization of the oligonucleotide to the target nucleic acid.

By “derived” is meant that the referred to nucleic acid is obtained directly from a target virus or indirectly as the product of a nucleic acid amplification, which product may be, for instance, an antisense RNA molecule which does not exist in the target virus.

By “capture probe” is meant at least one nucleic acid oligonucleotide that provides means for specifically joining a target sequence and an immobilized oligonucleotide due to base pair hybridization. A capture probe preferably includes two binding regions: a target sequence-binding region and an immobilized probe-binding region which are generally contiguous on the same oligonucleotide, although these regions may be present on distinct oligonucleotides and joined together by one or more linkers (see, e.g., Becker et al., “Method for Amplifying Target Nucleic Acids Using Modified Primers,” U.S. Pat. No. 6,130,038). A capture probe may alternatively be bound to a solid support by means of ligand-ligate binding pairs, such as avidin/biotin linkages.

By “immobilized probe” or “immobilized nucleic acid” is meant a nucleic acid that joins, directly or indirectly, a capture probe to an immobilized support. An immobilized probe is an oligonucleotide joined to a solid support that facilitates separation of bound target sequence from unbound material in a sample.

By “isolate” or “isolating” is meant that at least a portion of the target nucleic acid present in a test sample is concentrated within a reaction receptacle or on a reaction device or solid carrier (e.g., test tube, cuvette, microtiter plate well, nitrocellulose filter, slide or pipette tip) in a fixed or releasable manner so that the target nucleic acid can be purified without significant loss of the target nucleic acid from the receptacle, device or carrier.

By “purify” or “purifying” is meant that one or more components of the test sample are removed from one or more other components of the sample. Sample components to be purified may include viruses, nucleic acids or, in particular, target nucleic acids in a generally aqueous solution phase which may also include undesirable materials such as proteins, carbohydrates, lipids, non-target nucleic acid and/or labeled probes. Preferably, the purifying step removes at least about 70%, more preferably at least about 90% and, even more preferably, at least about 95% of the undesirable components present in the sample.

By “RNA and DNA equivalents” or “RNA and DNA equivalent bases” is meant RNA and DNA molecules having the same complementary base pair hybridization properties. RNA and DNA equivalents have different sugar moieties (i.e., ribose versus deoxyribose) and may differ by the presence of uracil in RNA and thymine in DNA. The differences between RNA and DNA equivalents do not contribute to differences in homology because the equivalents have the same degree of complementarity to a particular sequence.

By “consisting essentially of′ is meant that additional components, compositions or method steps that do not materially change the basic and novel characteristics of the present invention may be included in the compositions and methods of the present invention. Such characteristics include the ability to capture, amplify or selectively detect SARS-CoV derived nucleic acid in a test sample. Any component, composition or method step that has a material effect on the basic and novel characteristics of the present invention would fall outside of this term.

### Methods of Amplification

Amplification methods useful in connection with the present invention include Transcription-Mediated Amplification (TMA), Nucleic Acid Sequence-Based Amplification (NASBA), a reverse transcription form of the Polymerase Chain Reaction (RT-PCR), Strand Displacement Amplification (SDA), and amplification methods using self-replicating polynucleotide molecules and replication enzymes such as MDV-1 RNA and Q-beta enzyme. Methods for carrying out these various amplification techniques respectively can be found in the following: Kacian et al., U.S. Pat. No. 5,399,491; Davey et al., U.S. Pat. No. 5,554,517; van Gemen et al., “Quantification of Nucleic Acid,” U.S. Pat. No. 5,834,255; Mullis et al., “Process for Amplifying, Detecting, and/or Cloning Nucleic Acid Sequences Using a Thermostable Enzyme,” U.S. Pat. No. 4,965,188, Walker, “Strand Displacement Amplification,” U.S. Pat. No. 5,455,166; Chu et al., “Replicative RNA Reporter Systems,” U.S. Pat. No. 4,957,858; Stefano, “Nucleic Acid Structures with Catalytic and Autocatalytic Replicating Features and Methods of Use,” U.S. Pat. No. 5,472,840. Each of the foregoing references is hereby incorporated by reference herein.

In a highly preferred embodiment of the invention, nucleic acid sequences from SARS-CoV are amplified using a TMA protocol. According to this protocol, the reverse transcriptase which provides the DNA polymerase activity also possesses an endogenous RNase H activity. One of the amplification oligonucleotides used in this procedure contains a promoter sequence positioned upstream of a sequence that is complementary to one strand of a target nucleic acid that is to be amplified. In the first step of the amplification, a promoter-primer hybridizes to the target RNA of SARS-CoV at a defined site. Reverse transcriptase creates a complementary DNA copy of the target RNA by extension from the 3′ end of the promoter-primer. Following interaction of an opposite strand primer with the newly synthesized DNA strand, a second strand of DNA is synthesized from the end of the primer by reverse transcriptase, thereby creating a double-stranded DNA molecule. RNA polymerase recognizes the promoter sequence in this double-stranded DNA template and initiates transcription. Each of the newly synthesized RNA amplicons re-enters the TMA process and serves as a template for a new round of replication, thereby leading to an exponential expansion of the RNA amplicon. Since each of the DNA templates can make from about a 100 to about a 1000 copies of RNA amplicon, this expansion can result in the production of as many as 10 billion amplicons in less than one hour. The entire process is autocatalytic and is performed at a constant temperature.

### Structural Features of Amplification Oligonucleotides

As indicated above, a “primer” refers to an optionally modified oligonucleotide which is capable of hybridizing to a template nucleic acid and which has a 3′ end that can be extended by a DNA polymerase activity. The 5′ region of the primer may be non-complementary to the target nucleic acid. If the 5′ non-complementary region includes a promoter sequence, it is referred to as a “promoter-primer.” Those skilled in the art will appreciate that any oligonucleotide that can function as a primer (i.e., an oligonucleotide that hybridizes specifically to a target sequence and has a 3′ end capable of extension by a DNA polymerase activity) can be modified to include a 5′ promoter sequence, and thus could function as a promoter-primer. Similarly, any promoter-primer can be modified by removal of, or synthesis without, a promoter sequence and still function as a primer.

Nucleotide base moieties of primers may be modified (e.g., by the addition of propyne groups), as long as the modified base moiety retains the ability to form a non-covalent association with G, A, C, T or U, and as long as an oligonucleotide comprising at least one modified nucleotide base moiety or analog is not sterically prevented from hybridizing with a single-stranded nucleic acid. As indicated below in connection with the chemical composition of useful probes, the nitrogenous bases of primers in accordance with the invention may be conventional bases (A, G, C, T, U), known analogs thereof (e.g., inosine or “I” having hypoxanthine as its base moiety, (see, e.g., ROGER L. P. ADAMS ET AL., THE BIOCHEMISTRY OF THE NUCLEIC ACIDS (11^{th} ed. 1992)), known derivatives of purine or pyrimidine bases (e.g., N⁴-methyl deoxygaunosine, deaza- or aza-purines and deaza- or aza-pyrimidines, pyrimidine bases having substituent groups at the 5 or 6 position, purine bases having an altered or a replacement substituent at the 2, 6 or 8 positions, 2-amino-6-methylaminopurine, O⁶-methylguanine, 4-thio-pyrimidines, 4-amino-pyrimidines, 4-dimethylhydrazine-pyrimidines, and O⁴-alkyl-pyrimidines, (see, e.g., Cook et al., “Gapped 2′ Modified Oligonucleotides,” U.S. Pat. No. 5,623,065), and “abasic” residues where the backbone includes no nitrogenous base for one or more residues of the polymer (see Arnold et al., “Linking Reagents for Nucleotide Probes,” U.S. Pat. No. 5,585,481). Common sugar moieties that comprise the primer backbone include ribose and deoxyribose, although 2′-O-methyl ribose (OMe), (see Becker et al., U.S. Pat. No. 6,130,038), halogenated sugars, and other modified sugar moieties may also be used. Usually, the linking group of the primer backbone is a phosphorus-containing moiety, most commonly a phosphodiester linkage, although other linkages, such as, for example, phosphorothioates, methylphosphonates, and non-phosphorus-containing linkages such as peptide-like linkages found in “peptide nucleic acids” (PNA) also are intended for use in the assay disclosed herein.

### Useful Probe Labeling Systems and Detectable Moieties

Essentially any labeling and detection system that can be used for monitoring specific nucleic acid hybridization can be used in conjunction with the present invention. Included among the collection of useful labels are radiolabels, enzymes, haptens, linked oligonucleotides, chemiluminescent molecules and redox-active moieties that are amenable to electronic detection methods. Preferred chemiluminescent molecules include acridinium esters of the type disclosed by Arnold et al. in U.S. Pat. No. 5,283,174 for use in connection with homogenous protection assays, and of the type disclosed by Woodhead et al. in U.S. Pat. No. 5,656,207 for use in connection with assays that quantify multiple targets in a single reaction. Preferred electronic labeling and detection approaches are disclosed by Meade et al., “Nucleic Acid Mediated Electron Transfer,” U.S. Pat. Nos. 5,591,578, and Meade, “Detection of Analytes Using Reorganization Energy,” U.S. Pat. No. 6,013,170. Redox active moieties useful as labels in the present invention include transition metals such as Cd, Mg, Cu, Co, Pd, Zn, Fe and Ru.

Particularly preferred detectable labels for probes in accordance with the present invention are detectable in homogeneous assay systems (i.e., where, in a mixture, bound labeled probe exhibits a detectable change, such as stability or differential degradation, compared to unbound labeled probe). A preferred label for use in homogenous assays is a chemiluminescent compound (see, e.g., Woodhead et al., U.S. Pat. No. 5,656,207; Nelson et al., U.S. Pat. No. 5,658,737; and Arnold et al., “Homogenous Protection Assay,” U.S. Pat. No. 5,639,604). Particularly preferred chemiluminescent labels include acridinium ester (“AE”) compounds, such as standard AE or derivatives thereof, such as naphthyl-AE, ortho-AE, 1- or 3-methyl-AE, 2,7-dimethyl-AE, 4,5-dimethyl-AE, ortho-dibromo-AE, ortho-dimethyl-AE, meta-dimethyl-AE, ortho-methoxy-AE, ortho-methoxy(cinnamyl)-AE, ortho-methyl-AE, ortho-fluoro-AE, 1- or 3-methyl-ortho-fluoro-AE, 1- or 3-methyl-meta-difluoro-AE, and 2-methyl-AE.

In some applications, probes of the present invention are designed to undergo a detectable conformational change when the probes bind to the target nucleic acid. These probes preferably include a pair of interacting labels which cooperate when in close proximity to one another to produce a signal which is different from a signal produced from such labels when they are farther apart so that their cooperation is diminished. The labels may be associated with one or more molecular entities. Examples of such molecular entities include, but are not limited to, the probe constructions disclosed in the following: Morrison, “Competitive Homogeneous Assay,” U.S. Pat. No. 5,928,862 (bimolecular probes); Livak et al., “Hybridization Assay Using Self-Quenching Fluorescence Probe,” U.S. Pat. No. 6,030,787 (single molecule probes); and the self-hybridizing probes disclosed by Becker et al., “Molecular Torches,” U.S. Pat. No. 6,361,945 (“molecular torch” probes), and Tyagi et al., U.S. Pat. No. 5,925,517 (“molecular beacon” probes). These probes are useful in homogenous assays, especially real-time amplification procedures, since the probes only emit a detectable signal when they are hybridized to the target nucleic acid.

The molecular torch probes disclosed in U.S. Pat. No. 6,361,945 have distinct regions of self-complementarity, referred to as “the target binding domain” and “the target closing domain,” which are connected by a joining region and which hybridize to one another under predetermined hybridization assay conditions. When exposed to denaturing conditions, the complementary regions (which may be fully or partially complementary) of the molecular torch probe melt, leaving the target binding domain available for hybridization to a target sequence when the predetermined hybridization assay conditions are restored. And when exposed to strand displacement conditions, a portion of the target sequence binds to the target binding domain and displaces the target closing domain from the target binding domain. Molecular torch probes are designed so that the target binding domain favors hybridization to the target sequence over the target closing domain. The target binding domain and the target closing domain of a molecular torch probe include interacting labels (e.g., luminescent/quencher) positioned so that a different signal is produced when the molecular torch probe is self-hybridized as opposed to when the molecular torch probe is hybridized to a target nucleic acid, thereby permitting detection of probe:target duplexes in a test sample in the presence of unhybridized probe having a viable label or labels associated therewith.

The molecular beacon probes disclosed in U.S. Pat. No. 5,925,517 comprise nucleic acid molecules having a target complement sequence, an affinity pair (or nucleic acid arms or stems) holding the probe in a closed conformation in the absence of a target nucleic acid sequence, and a label pair that interacts when the probe is in a closed conformation. Hybridization of the target nucleic acid and the target complement sequence separates the members of the affinity pair, thereby shifting the probe to an open conformation. The shift to the open conformation is detectable due to reduced interaction of the label pair, which may be, for example, a fluorophore and a quencher (e.g., DABCYL and EDANS).

Different types of interacting labels can be used to determine whether a probe has undergone a conformational change. For example, the interacting labels may be a luminescent/quencher pair, a luminescent/adduct pair, a Förrester energy transfer pair or a dye dimer. More than one type of label may be present on a particular molecule.

A luminescent/quencher pair is made up of one or more luminescent labels, such as chemiluminescent or fluorescent labels, and one or more quenchers. Preferably, a fluorescent/quencher pair is used to detect a probe which has undergone a conformational change. A fluorescent label absorbs light of a particular wavelength, or wavelength range, and emits light with a particular emission wavelength, or wavelength range. A quencher dampens, partially or completely, signal emitted from an excited fluorescent label. Quenchers can dampen signal production from different fluorophores. For example, 4-(4′-dimethyl-amino-phenylaxo)benzoic acid (DABCYL) can quench about 95% of the signal produced from 5-(2′-aminoethyl)aminoaphthaline-1-sulfonic acid (EDANS), rhodamine and fluorescein.

Different numbers and types of fluorescent and quencher labels can be used. For example, multiple fluorescent labels can be used to increase signal production from an opened torch, and multiple quenchers can be used to help ensure that in the absence of a target sequence an excited fluorescent molecule produces little or no signal. Examples of fluorophores include acridine, fluorescein, sulforhodamine 101, rhodamine, EDANS, Texas Red, Eosine, Bodipy and lucifer yellow. See, e.g., Tyagi et al., Nature Biotechnology, 16:49-53 (1998). Examples of quenchers include DABCYL, Thallium, Cesium, and p-xylene-bis-pyridinium bromide.

A luminescent/adduct pair is made up of one or more luminescent labels and one or more molecules able to form an adduct with the luminescent molecule(s) and, thereby, diminish signal production from the luminescent molecule(s). The use of adduct formation to alter signals from a luminescent molecule using ligands free in solution is disclosed by Becker et al., “Adduct Protection Assay,” U.S. Pat. No. 5,731,148.

Förrester energy transfer pairs are made up of two labels where the emission spectra of a first label overlaps with the excitation spectra of a second label. The first label can be excited and emission characteristic of the second label can be measured to determine if the labels are interacting. Examples of Förrester energy transfer pairs include pairs involving fluorescein and rhodamine; nitrobenz-2-oxa-1,3-diazole and rhodamine; fluorescein and tetramethylrhodamine; fluorescein and fluorescein; IAEDANS and fluorescein; and BODIPYFL and BIODIPYFL.

A dye dimer is made up of two dyes that interact upon formation of a dimer to produce a different signal than when the dyes are not in a dimer conformation. Dye dimer interactions are disclosed by Packard et al., Proc. Natl. Sci. USA, 93:11640-11645 (1996).

Synthetic techniques and methods of bonding labels to nucleic acids and detecting labels are well known in the art. See, e.g., J. SAMBROOK ET AL., MOLECULAR CLONING: A LABORATORY MANUAL, ch. 10 (2d ed. 1989); Becker et al., U.S. Pat. No. 6,361,945; Tyagi et al., U.S. Pat. No. 5,925,517, Tyagi et al., “Nucleic Acid Detection Probes Having Non-FRET Fluorescence Quenching and Kits and Assays Including Such Probes,” U.S. Pat. No. 6,150,097; Nelson et al., U.S. Pat. No. 5,658,737; Woodhead et al., U.S. Pat. No. 5,656,207; Hogan et al., “Nucleic Acid Probes for Detection and/or Quantitation of Non-Viral Organisms,” U.S. Pat. No. 5,547,842; Arnold et al., U.S. Pat. No. 5,283,174; Kourilsky et al., “Method of Detecting and Characterizing a Nucleic Acid or Reactant for the Application of this Method,” U.S. Pat. No. 4,581,333; and Becker et al., U.S. Pat. No. 5,731,148.

### Chemical Composition of Probes

Probes in accordance with the present invention comprise polynucleotides or polynucleotide analogs and optionally may carry a detectable label or group of interacting labels covalently bonded thereto. Nucleosides or nucleoside analogs of the probe comprise nitrogenous heterocyclic bases, or base analogs, where the nucleosides are linked together, for example by phospohdiester bonds to form a polynucleotide. Accordingly, a probe may comprise conventional ribonucleic acid (RNA) and/or deoxyribonucleic acid (DNA), but also may comprise chemical analogs of these molecules. The “backbone” of a probe may be made up of a variety of linkages known in the art, including one or more sugar-phosphodiester linkages, peptide-nucleic acid bonds (referred to as “peptide nucleic acids” or “PNAs” as described by Nielsen et al., “Peptide Nucleic Acids,” U.S. Pat. No. 5,539,082), phosphorothioate linkages, methylphosphonate linkages or combinations thereof. Sugar moieties of the probe may be either ribose or deoxyribose, or similar compounds having known substitutions, such as, for example, 2′-O-methyl ribose and 2′ halide substitutions (e.g., 2′-F). The nitrogenous bases may be conventional bases (A, G, C, T, U), known analogs thereof (e.g., inosine or “I” (see ROGER L. P. ADAMS ET AL., THE BIOCHEMISTRY OF THE NUCLEIC ACIDS (11^{th} ed. 1992)), known derivatives of purine or pyrimidine bases (e.g., N⁴-methyl deoxygaunosine, deaza- or aza-purines and deaza- or aza-pyrimidines, pyrimidine bases having substituent groups at the 5 or 6 position, purine bases having an altered or a replacement substituent at the 2, 6 or 8 positions, 2-amino-6-methylaminopurine, O⁶-methylguanine, 4-thio-pyrimidines, 4-amino-pyrimidines, 4-dimethylhydrazine-pyrimidines, and O⁴-alkyl-pyrimidines (see Cook et al., U.S. Pat. No. 5,623,065) and “abasic” residues where the backbone includes no nitrogenous base for one or more residues of the polymer (see Arnold et al., U.S. Pat. No. 5,585,481). A probe may comprise only conventional sugars, bases and linkages found in RNA and DNA, or may include both conventional components and substitutions (e.g., conventional bases linked via a methoxy backbone, or a nucleic acid including conventional bases and one or more base analogs).

While oligonucleotide probes of different lengths and base composition may be used for detecting nucleic acids derived from SARS-CoV, preferred probes in this invention have lengths of up to 100 bases, and more preferably have lengths of up to 60 nucleotides. Preferred length ranges for the oligonucleotides of the present invention are from 10 to 100 bases in length, more preferably between 15 and 50 bases in length, and most preferably from 18 to 20, 25, 30 or 35 bases in length. However, probe sequences may also be provided in a nucleic acid cloning vector or transcript or other longer nucleic acid and still can be used for detecting nucleic acids derived from SARS-CoV.

### Selection of Amplification Oligonucleotides and Detection Probes Specific for SARS-CoV

Useful guidelines for designing amplification oligonucleotides and detection probes with desired characteristics are described herein. The optimal sites for amplifying and probing nucleic acid derived from SARS-CoV contain two, and preferably three, conserved regions each preferably at least about 15 bases in length and contained within about 200 bases of contiguous sequence. The degree of amplification observed with a set of amplification oligonucleotides depends on several factors, including the ability of the oligonucleotides to hybridize to their complementary sequences and their ability to be extended enzymatically. Because the extent and specificity of hybridization reactions are affected by a number of factors, manipulation of those factors will determine the exact sensitivity and specificity of a particular oligonucleotide, whether perfectly complementary to its target or not. The effects of varying assay conditions are known to those skilled in the art and are disclosed by Hogan et al., “Nucleic Acid Probes for Detection and/or Quantitation of Non-Viral Organisms,” U.S. Pat. No. 5,840,488.

The length of the target nucleic acid sequence and, accordingly, the length of amplification oligonucleotide and/or probe sequences can be important. In some cases, there may be several sequences from a particular target region, varying in location and length, which will yield amplification oligonucleotides or probes having the desired hybridization characteristics. While it is possible for nucleic acids that are not perfectly complementary to hybridize, the longest stretch of perfectly homologous base sequence will normally primarily determine hybrid stability.

Amplification oligonucleotides and probes should be positioned to minimize the stability of oligonucleotide:non-target nucleic acid hybrid, where the “non-target” is nucleic acid derived from a non-targeted organism or virus that contains a sequence similar to that of the target nucleic acid sequence. It is preferred that the amplification oligonucleotides and detection probes are able to distinguish between target and non-target sequences. In designing amplification oligonucleotides and probes, the differences in these Tₘ values should be as large as possible (e.g., at least 2° C. and preferably at least 5° C.).

The degree of non-specific extension (primer-dimer or non-target copying) can also affect amplification efficiency. For this reason, primers are selected to have low self- or cross-complementarity, particularly at the 3′ ends of the sequence. Long homopolymer tracts and high GC content are avoided to reduce spurious primer extension. Commercially available computer software can aid in this aspect of the design. Available computer programs include MacDNASIS™ 2.0 (Hitachi Software Engineering American Ltd.; San Francisco, Calif.) and OLIGO ver. 6.6 (Molecular Biology Insights; Cascade, Colo.).

Those having an ordinary level of skill in the art will appreciate that hybridization involves the association of two single strands of complementary nucleic acid to form a hydrogen bonded double strand. It is implicit that if one of the two strands is wholly or partially involved in a hybrid, then that strand will be less able to participate in formation of a new hybrid. By designing amplification oligonucleotides and probes so that substantial portions of the sequences of interest are single stranded, the rate and extent of hybridization may be greatly increased. If the target is an integrated genomic sequence, then it will naturally occur in a double-stranded form (as is the case with the product of a polymerase chain reaction amplification). These double-stranded targets are naturally inhibitory to hybridization with a probe and require denaturation prior to the hybridization step.

The rate at which a polynucleotide hybridizes to its target is a measure of the thermal stability of the target secondary structure in the target binding region. The standard measurement of hybridization rate is the C₀t_{1/2} which is measured as moles of nucleotide per liter multiplied by seconds. Thus, it is the concentration of probe multiplied by the time at which 50% of maximal hybridization occurs at that concentration. This value is determined by hybridizing various amounts of polynucleotide to a constant amount of target for a fixed time. The C₀t_{1/2} is found graphically by standard procedures familiar to those having an ordinary level of skill in the art.

### Preferred Amplification Oligonucleotides

Amplification oligonucleotides useful for conducting amplification reactions can have different lengths to accommodate the presence of extraneous sequences that do not participate in target binding, and that may not substantially affect amplification or detection procedures. For example, promoter-primers useful for performing amplification reactions in accordance with the invention have at least a minimal sequence that hybridizes to the target nucleic acid of SARS-CoV, and a promoter sequence positioned upstream of that minimal sequence. However, insertion of sequences between the target binding sequence and the promoter sequence could change the length of the primer without compromising its utility in the amplification reaction. Additionally, the lengths of the amplification oligonucleotides and detection probes are matters of choice as long as the sequences of these oligonucleotides conform to the minimal essential requirements for hybridizing the desired complementary sequence.

Particularly preferred amplification oligonucleotides of the present invention target RNA regions of SARS-CoV that are conserved relative to corresponding regions in the RNA of other coronaviruses. By “conserved” is meant that the region derived from SARS-CoV RNA targeted by the amplification oligonucleotide is at least about 60% homologous, preferably at least about 70% homologous, more preferably at least about 80% homologous, even more preferably at least about 90% homologous, and most preferably 100% homologous to the corresponding region derived from the RNA of other coronaviruses (e.g., HCoV-OC43 and HCoV-229E). Conserved regions of SARS-CoV RNA are preferably targeted by the amplification oligonucleotides of the present invention because it is expected that these regions will exhibit fewer mutations over time than regions having less sequence homology.

Perfect complementarity between the target binding region of an amplification oligonucleotide of the present invention and the target region of SARS-CoV RNA is not required, provided there is sufficient complementarity for the amplification oligonucleotide to bind to the target region under the amplification conditions selected. If the amplification oligonucleotide is to be extended by a polymerase, however, then the sequence of the amplification oligonucleotide should be designed so that its 3′ most base binds to its complementary base in the target sequence under the selected amplification conditions. This design feature would not be required where the amplification oligonucleotide is a promoter-primer containing a modification at or near the 3′ end of the “primer” or template binding sequence which reduces or blocks extension of the primer sequence by a polymerase. Blocked promoter-primers are disclosed by McDonough et al., U.S. Pat. No. 5,766,849.

Amplification oligonucleotides having target binding regions which bind to conserved regions of SARS-CoV RNA under selected amplification conditions were identified by comparing the sequences of the following GenBank accession numbers: NC_{—}004718 (SARS coronavirus, complete genome), AY278554 (SARS coronavirus CUHK-W1, complete genome), AY269391 (SARS coronavirus Vietnam strain 200300592 polymerase gene, partial cds), AF124990 (rat sialodacryoadenitis coronavirus RNA-directed RNA polymerase (pol) gene, partial cds), Z34093 (transmissible gastroenteritis virus (Purdue-115) mRNA for polymerase locus), AF304460 (human coronavirus 229E, complete genome), M95169 (avian infectious bronchitis virus pol protein, spike protein, small virion-associated protein, membrane protein, and nucleocapsid protein genes, complete cds), AF220295 (bovine coronavirus strain Quebec, complete genome), AF201929 (murine hepatitis virus strain 2, complete genome), M94356 (avian infectious bronchitis virus ORF1a (F1) and ORF1b (F2) genes, complete cds; S protein gene, partial cds; and unknown gene), M55148 (murine coronavirus open reading frame 1a (gene 1), complete cds and open reading frame 1b (gene 1), 3′ end), X69721 (human coronavirus 229E mRNA for RNA polymerase and proteases), AF124992 (porcine transmissible gastroenteritis virus RNA-directed RNA polymerase (pol) gene, partial cds), AF124989 (human coronavirus (strain OC43) RNA-directed RNA polymerase (pol) gene, partial cds), AF124987 (feline infectious peritonitis virus RNA-directed RNA polymerase (pol) gene, partial cds), AF124986 (canine coronavirus RNA-directed RNA polymerase (pol) gene, partial cds), AF124985 (bovine coronavirus RNA-directed RNA polymerase (pol) gene, partial cds), X51939 (mouse hepatitis virus RNA for viral polymerase open reading frame 1b), AJ011482 (porcine transmissible gastroenteritis virus minigenome), AJ311317 (avian infectious bronchitis virus (strain Beaudette CK) complete genomic RNA), Z30541 (avian infectious bronchitis virus mRNA for chimeric gene), AF208067 (murine hepatitis virus strain ML-10, complete genome), AJ271965 (transmissible gastroenteritis virus complete genome, genomic RNA), AF391542 (bovine coronavirus isolate BCoV-LUN, complete genome), AF391541 (bovine coronavirus isolate BCoV-ENT, complete genome), AF208066 (murine hepatitis virus strain Penn 97-1, complete genome), AF029248 (mouse hepatitis virus strain MHV-A59 C12 mutant, complete genome), Z69629 (infectious bronchitis virus RNA (defective RNA CD-61), AF207902 (murine hepatitis virus strain ML-11 RNA-directed RNA polymerase (orf1A), RNA-directed RNA polymerase (orf1B), non-structural protein (orf2A), hemagglutinin esterase protein (orf2B), spike glycoprotein precursor (orf3), non-structural protein (orf5A), envelope glycoprotein E (orf5B), matrix glycoprotein (orf6), and nucleocapsid protein (orf7) genes, complete cds), AF124991 (turkey coronavirus RNA-directed RNA polymerase (pol) gene, partial cds), AF124988 (porcine hemagglutinating encephalomyelitis virus RNA-directed RNA polymerase (pol) gene, partial cds).

In one embodiment of the present invention, a first oligonucleotide set is provided which comprises two or more oligonucleotides capable of amplifying a target region of nucleic acid derived from SARS-CoV under amplification conditions, where the target region is contained within the sequence of SEQ ID NO:8 or its complement. In a preferred mode, the first oligonucleotide set includes first and second oligonucleotides, each oligonucleotide being up to 100 bases in length. The first oligonucleotide of the first oligonucleotide set is preferably selected to bind to or extend through a target sequence contained within the sequence of SEQ ID NO:9 or its complement under amplification conditions. More preferably, the target sequence of the first oligonucleotide is selected from the following sequences and their complements: SEQ ID NO:11, SEQ ID NO:12, SEQ ID NO:13, SEQ ID NO:14, SEQ ID NO:15, SEQ ID NO:16, SEQ ID NO:17, SEQ ID NO:18 and SEQ ID NO:19. The second oligonucleotide of the first oligonucleotide set is preferably selected to bind to or extend through a target sequence contained within the sequence of SEQ ID NO:10 or its complement under amplification conditions. More preferably, the target sequence of the second oligonucleotide is selected from the following sequences and their complements: SEQ ID NO:20, SEQ ID NO:21 and SEQ ID NO:22.

In another embodiment of the present invention, a second oligonucleotide set is provided which comprises two or more oligonucleotides capable of amplifying target region of nucleic acid derived from SARS-CoV under amplification conditions, where the target region is contained within the sequence of SEQ ID NO:23 or its complement. In a preferred mode, the second oligonucleotide set includes first and second oligonucleotides, each oligonucleotide being up to 100 bases in length. The first oligonucleotide of the second oligonucleotide set is preferably selected to bind to or extend through a target sequence contained within the sequence of SEQ ID NO:24 or its complement under amplification conditions. More preferably, the target sequence of the first oligonucleotide is selected from the following sequences and their complements: SEQ ID NO:26 and SEQ ID NO:27. The second oligonucleotide of the second oligonucleotide set is preferably selected to bind to or extend through a target sequence contained within the sequence of SEQ ID NO:25 or its complement under amplification conditions. More preferably, the target sequence of the second oligonucleotide is selected from the following sequences and their complements: SEQ ID NO:28, SEQ ID NO:29, SEQ ID NO:30, SEQ ID NO:31, SEQ ID NO:32 and SEQ ID NO:33.

At least one of the amplification oligonucleotides of this set of amplification oligonucleotides may include a 5′ sequence which is recognized by a RNA polymerase or which enhances initiation or elongation by RNA polymerase. When included, the first amplification oligonucleotide preferably includes a T7 promoter sequence having the sequence SEQ ID NO:34.

Particularly preferred amplification oligonucleotides of the present invention target the 5′ leader sequence present in the genomic RNA or the 5′ leader sequence of at least one of the subgenomic mRNA sequences and/or a 3′ terminal sequence present in all SARS-CoV RNAs. Leader sequences and 3′ co-terminal sequences are preferred because these sequences are generally present in multiple copies in a test sample, thereby providing an inherent amplification of the target sequences which should result in better assay sensitivity. The leader sequences also contain a core sequence in the transcription regulating sequence (TRS), which is a conserved motif that can be targeted by at least one amplification oligonucleotide. If the TRS core sequence is targeted by an amplification oligonucleotide of the present invention, then the 3′ end of the amplification oligonucleotide preferably binds to the core sequence under amplification conditions and is extended therefrom.

In order to precisely define the bounds of the 5′ leader and the 3′ terminal sequences, various procedures are known to those skilled in the art for locating these sequences in cells infected with other coronaviruses. For example, SARS-CoV has been propagated in Vero cells. Total mRNAs from such cells may be isolated using standard methods well-known to those skilled in the art for the purification of polyadenylated mRNAs. The viral RNAs may be further enriched by target capture using capture probes homologous to the 3′ terminal sequences of the plus strand viral genome and then cloned using methods that preserve the 5′ terminal sequences. These methods include those in which cDNA synthesized from the viral RNA using oligo dT amplification oligonucleotides hybridized to the 3′ poly(A) tail. A tail is itself extended with a homopolymer tail prior to cloning.

Probes complementary to the 3′-most 50-100 nucleotides and the 5′-most 250 nucleotides can be used to identify clones that contain sequences from both regions. These clones may then be sequenced and compared to determine the exact sequences that comprise the 5′ leader sequences and 3′ terminal sequences that are present in each of the viral RNAs.

Alternatively, the polyadenylated mRNAs may be copied using reverse transcriptase and amplification oligonucleotides complementary to the 3′-most terminal nucleotides of the plus strand viral genome. The resulting cDNAs may be tailed at their 3′ ends with, for example, oligo C, and the resulting cDNAs amplified by PCR. The amplicons may then be separated by size by gel electrophoresis and sequenced.

Our approach was to identify the region in which the 5′ leader sequence is located by comparing 12 published SARS-CoV sequences having the following GenBank accession numbers: AY268049 (SARS coronavirus Taiwan RNA-directed RNA polymerase (pol) gene, partial cds), AY269391 (SARS coronavirus Vietnam strain 200300592 polymerase gene, partial cds), AY274119 (SARS coronavirus TOR2, complete genome), AY278487 (SARS coronavirus BJ02, partial genome), AY278488 (SARS coronavirus BJ0, complete genome), AY278489 (SARS coronavirus GZ01, partial genome), AY278490 (SARS coronavirus BJ03, partial genome), AY278491 (SARS coronavirus HKU-39849, complete genome), AY278554 (SARS coronavirus CUHK-W1, complete genome), AY278741 (SARS coronavirus Urbani, complete genome), AY279354 (SARS coronavirus BJ04, partial genome) and NC_{—}004718 (SARS coronavirus TOR2, complete genome).

Using the first sequence version available for the SARS TOR2 strain (GenBank accession number NC_{—}004718), we initially “walked” along the first 520 nucleotides of the SARS-CoV genome and selected all possible subsequences having lengths of 6 and 7 nucleotides. Each of these subsequences was then compared with the SARS coronavirus TOR2 genome sequence to identify perfectly matched sequences elsewhere in the genome. Those that yielded a number of matches in the expected range of 8 to 11 were examined to determine whether the subsequences were located within 50 nucleotides 5′ of the start of each potential gene identified in the annotated GenBank file for the first sequence version available for the SARS TOR2 strain (i.e., 21477 (spike glycoprotein), 25253, 25674, 26102 (small envelope protein E), 27059, 27258, 28105 (nucleocapsid protein) and 28115). (In the annotated GenBank file for the third sequence version available for the SARS TOR2 strain, released Mar. 24, 2004, the start of each potential gene is identified as follows: 21492 (spike glycoprotein); 25268 (orf3); 25689 (orf4); 26117 (small envelope protein E); 26398 (membrane glycoprotein M); 27074 (orf7); 27273 (orf8); 27638 (orf9); 27779 (orf10); 27864 (orf11); 28120 (nucleocapsid protein); 28130 (orf13); and 28583 (orf14).) Those subsequences located within 50 nucleotides of the start of the majority of potential gene sequences were potential candidates for the TRS core sequence of SARS-CoV.

We next examined sequences spanning from 100 nucleotides prior to the start codon through the start codon, as well as the initial untranslated region at the 5′ end, to verify the locations of the putative TRS and whether the TRS core sequence spanned a stretch more than 6 to 7 nucleotides in length. In each segment, we found sequences that were 7 to 10 nucleotides in length and which shared the nucleotide sequence of ua[a][a][a]cgaac (SEQ ID NO:38), where the brackets indicate additional nucleotides which may be present in the core sequence of the TRS. This core sequence spanned nucleotides 49 to 57 of version 1 of the SARS TOR2 genome sequence (nucleotides 64 to 72 of version 3 of this sequence).

When SARS-CoV RNAs are synthesized, the leader sequence in the 5′ untranslated region should be incorporated into the 5′ terminus of each genome RNA and subgenomic mRNA, although the leader sequences differ somewhat in each of the RNAs. Accordingly, a particularly preferred amplification oligonucleotide of the present invention binds to a target sequence contained within or complementary to a portion of the 5′ untranslated region that ends with the TRS core sequence under amplification conditions. In a preferred mode, a set of opposed amplification oligonucleotides is employed, each member of the set of amplification oligonucleotides binding to a distinct region of a leader sequence or its complement. In an alternative embodiment, the set of amplification oligonucleotides may include an amplification oligonucleotide which binds to a target sequence contained in a leader sequence, or its complement, and an amplification oligonucleotide which binds to a target sequence contained in the 3′ terminal gene, or its complement, under amplification conditions. The amplification oligonucleotides are preferably selected to minimize complementarity to sequences of non-targeted organisms or viruses.

In samples that contain infected cells, or material derived from infected cells, in addition to mature virus particles, the sensitivity of the assay may be enhanced by targeting at least one 5′ leader sequence and/or the 3′ terminal gene sequence that is present in each member of the set of subgenomic mRNAs that is produced in infected cells. Thus, by choosing amplification oligonucleotide sets that effect amplification of sequences found in one or more 5′ leader sequences and/or the 3′ terminal gene, amplification of more abundant targets and greater assay sensitivity may be achieved. Since these sequences are also present at the termini of the genomic RNA of the mature virus itself, additional target molecules from that source may also be present in the sample. In addition, opposed amplification oligonucleotides in which at least one amplification oligonucleotide is located in one or more of the 5′ leader sequences and an opposed amplification oligonucleotide is located in the 3′ terminal gene can be used to amplify the genomic RNA and the subgenomic mRNA sequences located between the opposed amplification oligonucleotides.

Amplification oligonucleotides of the present invention are preferably unlabeled but may include one or more reporter groups to facilitate detection of a target nucleic acid in combination with or exclusive of detection probe. A wide variety of methods are available to detect an amplified target sequence. For example, the nucleotide substrates or the amplification oligonucleotides can include a detectable label which is incorporated into newly synthesized DNA. The resulting labeled amplification product is then generally separated from the unused labeled nucleotides or amplification oligonucleotides and the label is detected in the separated product fraction. (See, e.g., Wu, “Detection of Amplified Nucleic Acid Using Secondary Capture probes and Test Kit,” U.S. Pat. No. 5,387,510.)

A separation step is not required, however, if a amplification oligonucleotide is modified by, for example, linking it to two dyes which form a donor/acceptor dye pair. The modified amplification oligonucleotide can be designed so that the fluorescence of one dye pair member remains quenched by the other dye pair member, so long as the amplification oligonucleotide does not hybridize to target nucleic acid, thereby physically separating the two dyes. Moreover, the amplification oligonucleotide can be further modified to include a restriction endonuclease recognition site positioned between the two dyes so that when a hybrid is formed between the modified amplification oligonucleotide and target nucleic acid, the restriction endonuclease recognition site is rendered double-stranded and available for cleavage or nicking by an appropriate restriction endonuclease. Cleavage or nicking of the hybrid then separates the two dyes, resulting in a change in fluorescence due to decreased quenching which can be detected as an indication of the presence of the target virus in the test sample. This type of modified amplification oligonucleotide is disclosed by Nadeau et al., “Detection of Nucleic Acids by Fluorescence Quenching,” U.S. Pat. No. 6,054,279.

Substances which can serve as useful detectable labels are well known in the art and include radioactive isotopes, fluorescent molecules, chemiluminescent molecules, chromophores, as well as ligands such as biotin and haptens which, while not directly detectable, can be readily detected by a reaction with labeled forms of their specific binding partners, e.g., avidin and antibodies, respectively.

Another approach is to detect the amplification product by hybridization with a detectably labeled probe and measuring the resulting hybrids in any conventional manner. In particular, the product can be assayed by hybridizing a chemiluminescent acridinium ester-labeled oligonucleotide probe to the target sequence, selectively hydrolyzing the acridinium ester present on unhybridized probe, and measuring the chemiluminescence produced from the remaining acridinium ester in a luminometer. (See, e.g., U.S. Pat. No. 5,283,174 and NORMAN C. NELSON ET AL., NONISOTOPIC PROBING, BLOTTING, AND SEQUENCING, ch. 17 (Larry J. Kricka ed., 2d ed. 1995).)

### Preferred Detection Probes

Another aspect of the present invention relates to oligonucleotides that can be used as, or incorporated into, detection probes for use in detecting the presence of nucleic acid derived from SARS-CoV in a test sample. Methods for amplifying a target nucleic acid sequence present in nucleic acid derived from SARS-CoV can include an optional further step of detecting amplicons. This procedure for detecting nucleic acid derived from SARS-CoV includes a step of contacting a test sample with a detection probe that preferentially hybridizes to the target nucleic acid sequence, or the complement thereof, under stringent hybridization conditions, thereby forming a probe:target hybrid that is stable for detection. Next, there is a step of determining whether the probe:target hybrid is present in the test sample as an indication of the presence or absence of nucleic acid derived from SARS-CoV in the test sample. The determining step may involve direct detection of the probe:target hybrid.

Detection probes useful for detecting nucleic acid sequences derived from SARS-CoV include a sequence of bases substantially complementary to a target nucleic acid sequence of SARS-CoV or its complement. Thus, probes of the present invention hybridize to one strand of a target nucleic acid sequence of SARS-CoV or the complement thereof. These probes may optionally have additional bases outside of the targeted nucleic acid region which may or may not be complementary to nucleic acid derived from SARS-CoV.

Preferred detection probes are sufficiently complementary to the target nucleic acid sequence, or its complement, to hybridize therewith under stringent hybridization conditions corresponding to a temperature of about 60° C. when the salt concentration is in the range of about 0.6-0.9 M. Preferred salts include lithium chloride, but other salts such as sodium chloride and sodium citrate also can be used in the hybridization solution. Examples of high stringency hybridization conditions are alternatively provided by 0.48 M sodium phosphate buffer, 0.1% sodium dodecyl sulfate, and 1 mM each of EDTA and EGTA at a temperature of about 60°, or by 0.6 M LiCl, 1% lithium lauryl sulfate (LLS), 60 mM lithium succinate and 10 mM each of EDTA and EGTA at a temperature of about 60° C.

Preferred detection probes and amplification oligonucleotides of the present invention are selected to target “conserved regions” in SARS-CoV RNA. Conserved regions are defined supra in the section entitled “Preferred Amplification Oligonucleotides” and were identified by us by comparing the published sequences of GenBank accession nos. AY274119 (SARS coronavirus TOR2, complete genome), NC_{—}004718 (SARS coronavirus, complete genome), NC_{—}001451 (avian infectious bronchitis virus, complete genome), NC_{—}003045 (bovine coronavirus, complete genome), NC_{—}002306 (transmissible gastroenteritis virus, complete genome), NC_{—}001846 (murine hepatitis virus, complete genome) and NC_{—}003436 (procine epidemic diarrhea virus, complete genome) and NC_{—}002645 (human coronavirus 229E, complete genome) and a partial sequence of AY278741 (SARS coronavirus Urbani, complete genome). Importantly, there should be sufficient variability in the region targeted by the probe to distinguish SARS-CoV RNA from the RNA of other coronaviruses (e.g., human respiratory pathogens HCoV-229E and HCoV-OC43, at a minimum) and which may be present in a test sample. Preferred probes were initially selected based on a comparison of the above-identified sequences. These preferred probes were selected to bind to all or part of a sequence selected from the group consisting of SEQ ID NO:1, SEQ ID NO:3, and their complements.

Particularly preferred detection probes of the present invention include a target binding portion having a base sequence comprising, consisting of, substantially corresponding to, or contained within a base sequence selected from the group consisting of SEQ ID NO:2, SEQ ID NO:4, SEQ ID NO:5 and SEQ ID NO:6, their complements, and the RNA equivalents. In another preferred embodiment, the entire base sequence of probes comprises, consists of, substantially corresponds to, or is contained within a base sequence selected from the group consisting of SEQ ID NO:2, SEQ ID NO:4, SEQ ID NO:5 and SEQ ID NO:6, their complements, and the RNA equivalents thereof.

Detection probes of the present invention are preferably from 10 to 100 bases in length, more preferably from 15 to 50 bases in length, and most preferably 18 to 20, 25, 30 or 35 bases in length. In a preferred embodiment, the probes have an at least 10 contiguous base region that is perfectly complementary to an at least 10 contiguous base region of the target sequence or its perfect complement. In a more preferred embodiment, the probes have an at least 15 contiguous base region that is perfectly complementary to an at least 15 contiguous base region of the target sequence or its perfect complement. In addition to the target binding portion, probes of the present invention may include one or more base regions that do not stably bind to the target nucleic acid or its complement under stringent hybridization conditions. An additional base region may be used, for example, as a capture tail (e.g., poly(A) tail) to isolate hybridized probe in a test sample, or a pair of additional base regions may be provided to facilitate a closed conformation (e.g., self-hybridized, stem-loop structure) when the probe is not bound to the target nucleic acid or its complement. In some cases, base regions used to facilitate the closed conformation of a probe in the absence of target and the target binding portion of the probe overlap.

Preferred probes of the present invention include one or more detectable labels. In one embodiment, an acridinium ester label is joined to the probe by means of a non-nucleotide linker. For example, detection probes can be labeled with chemiluminescent acridinium ester compounds that are attached via a linker substantially as described by Arnold et al. in U.S. Pat. Nos. 5,585,481 and 5,639,604, the contents of which are hereby incorporated by reference herein. Particularly preferred probes of this embodiment have a base sequence comprising, consisting of, substantially corresponding to, or contained within the following sequences, their complements, and the RNA equivalents thereof, and a non-nucleotide linker positioned between the nucleotides indicated below with an asterisk:

| | |
|---|---|
| ccttatgg(*)gttgggattatcc, | SEQ ID NO:2 |
| ccttatgggttg(*)ggattatcc, | SEQ ID NO:2 |
| ccttat(*)gggttgggattatcc, | SEQ ID NO:2 |
| tgcgtggattggct(*)ttgatgt, | SEQ ID NO:6 |
| tgcgtggattgg(*)ctttgatgt, | SEQ ID NO:6 |
| tgcgtggattg(*)gctttgatgt, | SEQ ID NO:6 |
| and | |
| tgcgtggatt(*)ggctttgatgt. | SEQ ID NO:6 |

In another embodiment of the present invention, the probes include at least one pair of interacting labels which cooperate when in close proximity to one another (i.e., their relative relationship to each other when the probe is hybridized to another nucleic acid) to produce a first signal that is detectably different from a second signal produced from such labels when they are farther apart (i.e., their relative relationship to each other when the probe is not hybridized to another nucleic acid), so that their cooperation is diminished. Preferred is a luminescent/quencher pair made up of one or more luminescent labels, such as chemiluminescent or fluorescent labels, and one or more quenchers.

An example of a probe according to the present invention that is designed to assume differently detectable conformations, depending on whether the probe is bound to a nucleic acid (i.e., the target nucleic acid or its complement), has the following base sequence:

| | |
|---|---|
| ccgugcguggauuggcuuucacgg. | SEQ ID NO:7 |

The probe is fully comprised of 2′-O-methyl ribonucleotides, and the underlined portions of the sequence indicate the complementary arms that hybridize to each other under stringent hybridization conditions in the absence of target, thereby forming a stem-loop structure. This probe favors hybridization to the target. The target binding portion of this probe is the fully 2′-O-methyl ribonucleotide equivalent of the sequence as SEQ ID NO:4, thus the 5′ arm and the target binding portion of this probe overlap by four bases. Other probes described herein could be readily modified by those skilled in the art to be dual conformation probes. Since the two basic conformations (i.e., self-hybridized or hybridized to another nucleic acid) of dual conformation probes are differently detectable in a test sample, they are particularly useful in real-time amplification procedures in which the amount of amplicon present in a test sample is monitored during amplification of the target sequence.

For improved sensitivity, preferred methods of the present invention use detection probes targeting multiple regions present in or derived from the SARS-CoV RNA genome or they target one or more of the subgenomic mRNA 5′ leader sequences and/or a 3′ terminal sequence shared by all subgenomic mRNA sequences. If one or more leader sequences are targeted by the probes, then the target binding portion of each probe preferably includes a base sequence that hybridizes to all or a portion of the TRS core sequence.

In another preferred embodiment, a method is provided in which detection probes according to the present invention bind to a target sequence present in amplicon generated using an amplification oligonucleotide or set of amplification oligonucleotides, where at least one of the amplification oligonucleotides binds to the genomic RNA 5′ leader sequence or, preferably, to one of the subgenomic mRNA 5′ leader sequences of SARS-CoV under amplification conditions. In a preferred mode, at least one member of the set of amplification oligonucleotides targets a sequence comprising the TRS core sequence or its complement or a sequence contained in the 3′ terminal gene or its complement.

As indicated above, any number of different backbone structures can be used as a scaffold for the nucleobase sequences of the invented detection probes. In certain highly preferred embodiments, the probe sequences used include a methoxy backbone, or at least one methoxy linkage in the nucleic acid backbone.

### Preferred Helper Probes

Helper probes can be used in the methods of the present invention to facilitate hybridization of detection probes to their intended target nucleic acids, so that the detection probes more readily form probe:target nucleic acid duplexes than they would in the absence of helper probes. (Helper probes are disclosed by Hogan et al., “Means and Method for Enhancing Nucleic Acid Hybridization,” U.S. Pat. No. 5,030,557.) Each helper probe contains an oligonucleotide that is sufficiently complementary to a target nucleic acid sequence to form a helper probe:target nucleic acid duplex under stringent hybridization assay conditions. The stringent hybridization assay conditions employed with a given helper probe are determined by the conditions used for preferentially hybridizing the associated detection probe to the target nucleic acid.

Regions of single-stranded RNA and DNA can be involved in secondary and tertiary structures even under stringent hybridization assay conditions. Such structures can sterically inhibit or block hybridization of a detection probe to a target nucleic acid. Hybridization of the helper probe to the target nucleic acid alters the secondary and tertiary structure of the target nucleic acid, thereby rendering the target region more accessible by the detection probe. As a result, helper probes enhance the kinetics and/or the melting temperature of detection probe:target duplexes. Helper probes are generally selected to hybridize to nucleic acid sequences located near the target region of the detection probe.

Helper probes of the present invention comprise oligonucleotides which bind to target sequences contained within SARS-CoV-derived nucleic acid under stringent hybridization conditions. The helper probes are preferably substantially complementary to their intended target sequences. Detection probes and their associated helper probes are designed to hybridize to different target sequences contained within the same target nucleic acid. The helper probes of the present invention are preferably oligonucleotides up to 100 bases in length, more preferably from 12 to 50 bases in length, and most preferably from 18 to 35 bases in length. The helper probes are preferably at least about 90% complementary to, and more preferably perfectly complementary to, their corresponding target regions.

### Selection and Use of Capture Probes

Preferred capture probes includes a base sequence that is complementary to a SARS-CoV-derived target sequence that is covalently attached to a “tail” portion (e.g., a base sequence) that serves as a target for immobilization on a solid support. Any backbone to link the nucleobase units of a capture probe may be used. In certain preferred embodiments the capture probe includes at least one methoxy linkage in the backbone. When the tail portion is a base sequence (e.g., a poly(T) sequence), it is preferably positioned at the 3′ end of the capture probe and can bind to a substantially complementary polynucleotide to provide a means for capturing bound SARS-CoV-derived nucleic acid in preference to other components in the test sample.

Although any base sequence that hybridizes to a complementary base sequence may be used in the tail sequence, it is preferred that the hybridizing sequence span a length of about 5-50 nucleotide residues. Particularly preferred tail sequences are substantially homopolymeric, containing about 10 to about 40 nucleotide residues, or more preferably about 14 to about 30 residues. A capture probe according to the present invention may include a first sequence that specifically binds a SARS-CoV target nucleic acid, and a second sequence that specifically binds an oligo(dT) stretch immobilized to a solid support.

A preferred assay for determining the presence of SARS-CoV in a test sample includes the steps of capturing a SARS-CoV target nucleic acid with a capture probe, amplifying a target region present in the target nucleic acid using at least two amplification oligonucleotides, and detecting the amplified nucleic acid by first hybridizing a detection probe to a target sequence contained within the amplified nucleic acid and then detecting the formation of a probe:target hybrid as an indication of the presence of SARS-CoV in the test sample. Preferred capture probes target a sequence present in a 5′ leader sequence or the shared 3′ terminal sequence of all subgenomic mRNA sequences.

The capturing step of this assay preferably employs a capture probe that hybridizes to a target sequence present in SARS-CoV-derived nucleic acid under hybridization conditions and includes a tail portion that serves as one component of a binding pair, such as a ligand (e.g., a biotin-avidin binding pair) that allows the target nucleic acid to be separated from other components of the sample. The tail portion of the capture probe is preferably a base sequence that hybridizes to a complementary sequence immobilized on a solid support particle. Preferably, the capture probe and the target nucleic acid are contacted in solution to take advantage of solution phase hybridization kinetics. Hybridization produces a capture probe:target complex which can then be immobilized through hybridization of the tail portion of the capture probe with an immobilized probe having a substantially complementary base sequence. What results is a complex comprising the target nucleic acid, the capture probe and the immobilized probe. The immobilized probe preferably contains a repetitious sequence (e.g., poly(dAdT)) or a homopolymeric sequence (e.g., poly(dA)), which is complementary to the tail sequence (e.g., poly(dTdA) or poly(dT)) and is attached to a solid support. The capture probe may also contain “spacer” residues, such as one or more nucleotides, located between the target binding sequence and the tail sequence of the capture probe which do not function to bind the target nucleic acid or the immobilized probe. Any solid support may be used for immobilizing target:capture probe complex. Useful supports may be either matrices or particles free in solution (e.g., nitrocellulose, nylon, glass, polyacrylate, mixed polymers, polystyrene, silane polypropylene and, preferably, magnetically attractable particles). Methods of attaching an immobilized probe to the solid support are well known. The solid support is preferably a particle which can be retrieved from solution using standard methods (e.g., centrifugation, magnetic attraction, and the like). Preferred solid supports are paramagnetic, monodisperse particles of uniform size ± about 5%.

Retrieving the target:capture probe:immobilized probe complex (“the complex”) in a test sample effectively concentrates the target nucleic acid (relative to its concentration in the test sample) and separates the target nucleic acid from amplification inhibitors which may be present in the test sample. The captured target may be washed one or more times, thereby purifying the target nucleic acid. This can be done by, for example, resuspending the particles with the attached complex in a washing solution and then retrieving the particles with the attached complex from the washing solution as described above. In a preferred embodiment, the capturing step takes place by sequentially hybridizing the capture probe with the target nucleic acid and then adjusting the hybridization conditions to permit hybridization of the tail sequence with an immobilized probe. See, e.g., Weisburg et al., “Two-Step Hybridization and Capture of a Polynucleotide,” U.S. Pat. No. 6,110,678. After the capturing step and any optional washing steps have been completed, a target sequence can then be amplified. To limit the number of handling steps, the target nucleic acid optionally can be amplified without releasing it from the capture probe.

In a preferred embodiment of the present invention, the capture probes are selected to target conserved regions of coronavirus RNA. What constitutes a conserved region and how a conserved region of coronavirus RNA can be identified are discussed supra in the sections entitled “Preferred Amplification Oligonucleotides” and “Preferred Detection Probes.” Preferred capture probes of the present invention were identified by comparing sequences published in GenBank and having the following GenBank accession numbers: AY278741 (SARS coronavirus Urbani, complete genome), AF124990 (rat sialodacryoadenitis coronavirus RNA-directed RNA polymerase (pol) gene, partial cds), AF353511 (porcine epidemic diarrhea virus strain CV777, complete genome), AF304460 (human coronavirus 229E, complete genome), M95169 (avian infectious bronchitis virus pol protein, spike protein, small virion-associated protein, membrane protein, and nucleocapsid protein genes, complete cds), AF220295 (bovine coronavirus strain Quebec, complete genome), AF201929 (murine hepatitis virus strain 2, complete genome), M94356 (avian infectious bronchitis virus ORF1a (F1) and ORF1b (F2) genes, complete cds; S protein gene, partial cds; and unknown gene), M55148 (murine coronavirus open reading frame 1a (gene 1), complete cds and open reading frame 1b (gene 1), 3′ end), X69721 (human coronavirus 229E mRNA for RNA polymerase and proteases), AF124992 (porcine transmissible gastroenteritis virus RNA-directed RNA polymerase (pol) gene, partial cds), AF124989 (human coronavirus (strain OC43) RNA-directed RNA polymerase (pol) gene, partial cds), AF124987 (feline infectious peritonitis virus RNA-directed RNA polymerase (pol) gene, partial cds), AF124986 (canine coronavirus RNA-directed RNA polymerase (pol) gene, partial cds), AF124985 (bovine coronavirus RNA-directed RNA polymerase (pol) gene, partial cds), X51939 (mouse hepatitis virus RNA for viral polymerase open reading frame 1b), AJ01482 (porcine transmissible gastroenteritis virus minigenome), AJ311317 (avian infectious bronchitis virus (strain Beaudette CK) complete genomic RNA), Z30541 (avian infectious bronchitis virus mRNA for chimeric gene), AF208067 (murine hepatitis virus strain ML-10, complete genome), AJ271965 (transmissible gastroenteritis virus complete genome, genomic RNA), AF391542 (bovine coronavirus isolate BCoV-LUN, complete genome), AF391541 (bovine coronavirus isolate BCoV-ENT, complete genome), AF208066 (murine hepatitis virus strain Penn 97-1, complete genome), AF029248 (mouse hepatitis virus strain MHV-A59 C12 mutant, complete genome), Z69629 (infectious bronchitis virus RNA (defective RNA CD-61), AF207902 (murine hepatitis virus strain ML-11 RNA-directed RNA polymerase (orf1A), RNA-directed RNA polymerase (orf1B), non-structural protein (orf2A), hemagglutinin esterase protein (orf2B), spike glycoprotein precursor (orf3), non-structural protein (orf5A), envelope glycoprotein E (orf5B), matrix glycoprotein (orf6), and nucleocapsid protein (orf7) genes, complete cds), AF124991 (turkey coronavirus RNA-directed RNA polymerase (pol) gene, partial cds), AF124988 (porcine hemagglutinating encephalomyelitis virus RNA-directed RNA polymerase (pol) gene, partial cds). The preferred capture probes were selected to bind to all or part of a sequence selected from the group consisting of SEQ ID NO:35, SEQ ID NO:36 and SEQ ID NO:37, and their complements. Particularly preferred capture probes of the present invention include a target binding portion having a base sequence comprising, consisting of, substantially corresponding to, or contained within a base sequence selected from the group consisting of SEQ ID NO:35, SEQ ID NO:36 and SEQ ID NO:37, their complements, and the RNA equivalents thereof. The preferred capture probes were also designed to have a flexible 3′ tail comprising the following sequence:

| | |
|---|---|
| tttaaaaaaaaaaaaaaaaaaaaaaaaaaaaaa. | SEQ ID NO:39 |

In some cases, it may be advantageous to isolate the virus particles themselves using immunological methods. The prior art indicates that antibodies specific to SARS-CoV are produced by infected patients. These antibodies, or antibodies with equivalent specificities produced in animals or using monoclonal antibody production methods, can be used to isolate, concentrate and purify the virus particles by binding to them and allowing them to be removed from the sample. The antibodies can be bound to solid supports to facilitate removal of the virus/antibody complex, either directly or through a variety of ligand/ligate binding reactions involving binding pairs such as avidin/biotin or second antibodies that bind to the first, virus-specific, antibody. Other ligand/ligate pairs for use in bioassays are known to those skilled in the art that can be readily employed in such methods to isolate, concentrate, and purify SARS-CoV particles from samples prior to testing in nucleic acid assays. In addition to the use of solid supports, methods involving immunoprecipitation or partitioning of antigen/antibody complexes into an immiscible liquid phase are known in the art and can be employed.

Other methods known in the art may also be used to purify SARS Co-V from samples prior to testing using the methods set forth herein. These include centrifugation of samples to remove cellular elements, debris, and other components larger than the virus particles followed by centrifugation at higher speeds to sediment the virus particles themselves. Sedimentation of the virus particles may be aided by precipitants such as polytethylene glycol that are commonly employed for this purpose. Adsorption of the virus onto solid matrices, ultrafiltration, gel filtration, density gradient and isopycnic centrifugion, and polymer phase separation are other methods of virus purification that may be employed to isolate, concentrate and purify the virus prior to testing.

### Diagnostic Systems for Detecting SARS-CoV Nucleic Acid

The present invention also contemplates diagnostic systems in kit form. A diagnostic system of the present invention may include a kit which contains, in an amount sufficient for at least one assay, any of the detection probes, capture probes and/or amplification oligonucleotides of the present invention in a packaging material. Typically, the kits will also include instructions recorded in a tangible form (e.g., contained on paper or an electronic medium) for using the packaged probes and/or amplification oligonucleotides in an amplification and/or detection assay for determining the presence or amount of SARS-CoV in a test sample. In addition, helper probes may be included in the kits.

The various components of the diagnostic systems may be provided in a variety of forms. For example, the required enzymes, the nucleotide triphosphates, the detection probes and/or amplification oligonucleotides may be provided as a lyophilized reagent. These lyophilized reagents may be pre-mixed before lyophilization so that when reconstituted they form a complete mixture with the proper ratio of each of the components ready for use in the assay. In addition, the diagnostic systems of the present invention may contain a reconstitution reagent for reconstituting the lyophilized reagents of the kit. In preferred kits for amplifying target nucleic acid derived from SARS-CoV, the enzymes, nucleotide triphosphates and required cofactors for the enzymes are provided as a single lyophilized reagent that, when reconstituted, forms a proper reagent for use in the present amplification methods. In these kits, a lyophilized amplification oligonucleotide reagent may also be provided. In other preferred kits, lyophilized probe reagents are provided.

Typical packaging materials would include solid matrices such as glass, plastic, paper, foil, micro-particles and the like, capable of holding within fixed limits detection probes, capture probes, helper probes and/or amplification oligonucleotides of the present invention. Thus, for example, the packaging materials can include glass vials used to contain sub-milligram (e.g., picogram or nanogram) quantities of a contemplated probe or amplification oligonucleotide, or they can be microtiter plate wells to which probes or amplification oligonucleotides of the present invention have been operatively affixed, i.e., linked so as to be capable of participating in an amplification and/or detection method of the present invention.

The instructions will typically indicate the reagents and/or concentrations of reagents and at least one assay method parameter which might be, for example, the relative amounts of reagents to use per amount of sample. In addition, such specifics as maintenance, time periods, temperature and buffer conditions may also be included.

The diagnostic systems of the present invention contemplate kits having any of the detection probes, capture probes and/or amplification oligonucleotides described herein, whether provided individually or in one of the preferred combinations described above, for use in amplifying and/or determining the presence or amount of SARS-CoV in a test sample.

### EXAMPLES

Examples are provided below illustrating different aspects and embodiments of the invention. Skilled artisans will appreciate that these examples are not intended to limit the invention to the specific embodiments described therein.

### Example 1

### Sensitivity of Real-Time SARS-CoV Assay

In this experiment, we tested the sensitivity of a SARS-CoV assay system targeting a SARS-CoV RNA transcript mapping to the replicase gene (“the trancript”). The assay system included a target capture step for isolating the transcript (see Weisburg et al., U.S. Pat. No. 6,110,678), an amplification step employing two sets of primers and promoter-primers in a Transcription-Mediated Amplification (TMA) procedure (see Kacian et al, U.S. Pat. No. 5,399,491), and a detection step for detecting the production of amplicon with a molecular beacon probe in real-time (see Tyagi et al., U.S. Pat. No. 5,925,517). The oligonucleotides of this experiment were synthesized using standard phosphoramidite chemistry, various methods of which are well known in the art. See, e.g., Caruthers et al., Methods in Enzymol., 154:287 (1987). Oligonucleotide synthesis can be or was performed using an Expedite™ 8909 Nucleic Acid Synthesizer (Applied Biosystems, Foster City, Calif.). The molecular beacon probe was synthesized to include interacting fluoroscein and DABCYL labels using 3′-DABCYL CPG (Glen Research Corporation, Sterling, Va.; Cat. No. 20-5912-14) and fluorescein phosphoramidite (BioGenex, San Ramon, Calif.; Cat. No. BTX-3008-01). Reactions were performed on 96 well plates and the amplification and detection steps were carried out on a DNA Engine Opticon® Continuous Fluorescence Detection System (MJ Research, Inc., Watertown, Mass.).

The transcript was initially diluted in a transcript buffer (790 mM N-2-hydroxyethelpiperazine-N′-2-ethanesulfonic acid (HEPES), 230 mM succinic acid, 10% (w/v) LLS, 680 mM LiOH and 0.03% (w/v) Foam Ban (Ultra Additives Incorporated, Boomfield, N.J.; Cat. No. MS-575)), and 30 μL aliquots of the transcript buffer containing 0, 10, 100, 1000, 10,000 or 100,000 copies of the transcript were provided to the tubes of Ten-Tube Units (Gen-Probe Incorporated, San Diego, Calif.; Cat. No. TU0022). There were two replicates for each copy number. We then added 100 μL of a target capture reagent identical to the transcript buffer, and further containing about 10 μg Sera-Mag™ MG-CM Carboxylate Modified (Seradyn, Inc., Indianapolis, Ind.; Cat. No. 24152105-050250), 1 micron, super-paramagnetic particles having a covalently bound oligo(dT)₁₄, to each tube of the Ten-Tube Units (“TTUs”). The target capture reagent was spiked with a target capture probe consisting of a target binding portion having the sequence of SEQ ID NO:37 and a 3′ tail having the sequence of SEQ ID NO:39 to a concentration of 4 pmol/mL in the target capture reagent. The TTUs were then covered and vortexed for 10 to 20 seconds, incubated in a 60° C. water bath for 20 minutes to permit hybridization of the target binding portion of the capture probe to the transcript, and cooled at room temperature for 15 minutes to facilitate hybridization of the oligo(dA)₃₀ sequence of the tail portion of the capture probe to oligo(dT)₁₄ bound to the magnetic particles. (The tail portion includes a 5′-ttt-3′ spacer sequence interposed between the target binding portion and the oligo(dA)₃₀ sequence to make the capture probe more flexible for binding to the immobilized oligo(dT)₁₄.) Following cooling of the samples, a DTS™ 1600 Target Capture System (Gen-Probe; Cat. No. 5202) was used to isolate and wash the magnetic particles. The DTS 1600 Target Capture System has a test tube bay for positioning TTUs and applying a magnetic field thereto. The TTUs were placed in the test tube bay on the DTS 1600 Target Capture System for about 5 minutes in the presence of the magnetic field to isolate the magnetic particles within the tubes, after which the sample solutions were aspirated from the TTUs. Each tube was then provided with 1 mL of a wash buffer (10 mM HEPES, 6.5 mM NaOH, 1 mM EDTA, 0.3% (v/v) ethanol, 0.02% (w/v) methyl-paraben, 0.01% (w/v) propyl-paraben, 150 mM NaCl, 0.1% (w/v) sodium lauryl sulfate, and 4 M NaOH to pH 7.5), covered and vortexed for 10 to 20 seconds to resuspend the magnetic particles. The TTUs were returned to the test tube bay on the DTS 1600 Target Capture System and allowed to stand at room temperature for about 5 minutes before the wash buffer was aspirated. The wash steps were repeated using 100 μL instead of 1 mL of the wash buffer. Following washing, 40 μL purified water was added to each tube before the TTUs were covered, vortexed for 1 minute and incubated at 60° C. for 5 minutes to elute transcript off the magnetic particles. The tubes were again placed in the test tube bay on the DTS 1600 Target Capture System and allowed to stand at room temperature for about 5 minutes in order to separate the magnetic particles from the eluted transcript.

For the amplification step, 20 μL sample aliquots were transferred from the tubes (without magnetic particles) to reaction wells of a 96 well plate containing 20 μL of an amplification reagent (4.616 g Trizma® base buffer, 2.364 g Trizma hydrochloride buffer, 43 mL MgCl₂, 1 M solution, 3.474 g KCl₂, 66.6 mL glycerol, 0.022g zinc acetate, 20 mL dATP, 100 mM solution, 20 mL dCTP, 100 mM solution, 20 mL dGTP, 100 mM solution, 20 mL dTTP, 100 mM solution, 0.4 mL ProClin 300 Preservative (Supelco, Bellefonte, Pa.; Cat. No. 48126), 40 mL ATP, 325 mM solution, 24.6 mL CTP, 325 mM solution, 40 mL GTP, 325 mM solution, 24.6 mL UTP, 325 mM solution, purified water bringing total volume to 85 L, and 6 M HCl to pH 8.2) spiked with the primers and molecular beacon probe comprised of 2′-O-methyl ribonucleotides (SEQ ID NO:7) so that the final concentration of each primer and the probe in each well was 150 pmol/mL and 200 pmol/mL, respectively. The primers of this reaction had the following nucleotide sequences, where the promoter-primers further contained the 5′ promoter sequence of SEQ ID NO:34:

| | |
|---|---|
| tctagttgcatgacagccctc, | SEQ ID NO:40 |
| (T7 promoter-primer) | |
| ccacagcatctctagttgcatg, | SEQ ID NO:41 |
| (T7 promoter-primer) | |
| ttaccctaatatgtttatcacc, | SEQ ID NO:42 |
| (non-T7 primer) | |
| and | |
| gtcaatggttaccctaatatgtt. | SEQ ID NO:43 |
| (non-T7 primer) | |

The plates were then loaded onto the DNA Engine Opticon Continuous Fluorescence Detection System and incubated at 60° C. for 5 minutes to permit hybridization of the T7 promoter-primers to the transcript, before lowering the temperature to 37° C. for one minute. At this point, 20 μL of an enzyme reagent (70 mM N-acetyl-L-cysteine (NALC), 10% (v/v) TRITON® X-102 detergent, 16 mM HEPES, 3 mM EDTA, 0.05% (w/v) sodium azide, 20 mM Trizma base, 50 mM KCl₂, 20% (v/v) glycerol, 150 mM trehalose, 4M NaOH to pH 7, 224 RTU/μL Moloney murine leukemia virus (“MMLV”) reverse transcriptase, and 140 U/μL T7 RNA polymerase, where one “unit” of activity is defined as the synthesis and release of 5.75 fmol cDNA in 15 minutes at 37° C. for MMLV reverse transcriptase, and the production of 5.0 fmol RNA transcript in 20 minutes at 37° C. for T7 RNA polymerase) was added to each reaction well, the plate was vortexed for 5 to 10 seconds and then reloaded on the instrument. The DNA Engine Opticon Continuous Fluorescence Detection System was programed to heat the plate at 42.5° C. for 15 minutes before taking a first fluorescent reading. An additional 99 fluorescent readings were taken at 26 second intervals at an essentially constant temperature of 42.5° C. for a total of 100 fluorescent readings before the amplification reaction was completed.

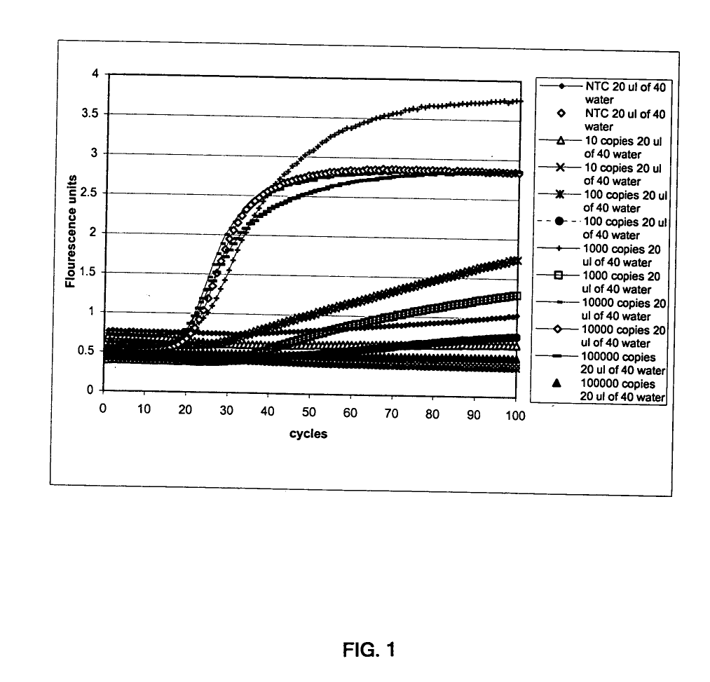

Detection in this assay system depended upon a conformational change in the molecular beacon probes as they hybridized to amplicon, thereby resulting in the emission of detectable fluorescent signals. As long as the molecular beacon probes maintained a hairpin configuration, i.e., they were not hybridized to an amplification product of the transcript, fluorescent emissions from the fluoroscein labels were generally quenched by the DABCYL labels. But as more of the molecular beacon probes hybridized to amplicon in the reaction wells, there was increase in detectable fluorescent signals. Thus, fluorescent emissions that increased over time provided an indication of active amplification of the target region of the transcript. Software provided with the DNA Engine Opticon Continuous Fluorescence Detection System was used to analyze results obtained using the molecular probes of the experiment, and the results are illustrated in the graph of FIG. 1, which shows fluorescence units detected from each reaction well on the y-axis versus the number of time cycles on the x-axis. A signal which rose above background (in the range of about 0.4 to 0.7 fluorescence units) and within 40 time cycles was considered to be a positive amplification. Using these criteria, it was determined that one of the reactions had a 100 copy sensitivity, and all of the reactions having at least 1000 copies of transcript were positive.

We note that in a separate experiment, molecular beacon probes having the following 2′-O-methyl ribonucleotide sequences did not appreciably hybridize to transcript amplicon in a real-time amplification assay using the primer sets of this experiment:

| | |
|---|---|
| ccgucgucacguucgugcgacgg, | SEQ ID NO:44 |
| and | |
| ccgacugauguagagggcugucgg. | SEQ ID NO:45 |

It is possible that these molecular beacon probes could detectably hybridize to target amplicon derived from the transcript in an optimized assay, or that the target binding portions of these probes could be incorporated into linear detection probes that would detectably hybridize to the targeted amplicon.

### Example 2

### Specificity and Sensitivity of SARS-CoV Assay

This experiment was designed to evaluate the specificity and sensitivity of a SARS-CoV assay system targeting a SARS-CoV RNA transcript mapping to the replicase gene (“the trancript”). Viral nucleic acid from human coronavirus strain 229E (“HcoV”), human immunodeficiency virus (“HIV”), hepatitis C virus (“HCV”), hepatitis B virus (“HBV”), and parvovirus were included to assess the cross-reactivity of this assay system. The assay system included the capture probe, target capture reagent, materials, instrumentation, and protocol of Example 1, an amplification step employing the primer/promoter-primer sets of Example 1 in a TMA reaction, and a detection step for detecting the production of amplicon with an acridinium-ester (AE)-labeled probe in a Hybridization Protection Assay (Arnold et al., U.S. Pat. No. 5,283,174). As above, the oligonucleotides of this experiment were synthesized using standard phosphoramidite chemistry, various methods of which are well known in the art. Oligonucleotide synthesis can be or was performed using an Expedite™ 8909 Nucleic Acid Synthesizer. The SARS-CoV detection probe had the base sequence of SEQ ID NO:46 ugcguggauuggcuuugaugt and a 2-methyl-AE label (the “glower”) joined to the probe by means of a non-nucleotide linker positioned between nucleotides 14 and 15 (see Arnold et al. in U.S. Pat. Nos. 5,585,481 and 5,639,604). With the exception of the 3′ most nucleotide, which was a deoxynucleotide, all of the nucleotides of the detection probe were 2′-O-methyl ribonucleotides. To confirm that the conditions were sufficient to support amplification, each sample included an internal control derived from HIV nucleic acid and oligonucleotides for amplifying and detecting amplicon of the internal control. The oligonucleotide used to detect amplicon of the internal control included an ortho-fluoro-AE label (the “flasher”) joined to the oligonucleotide by means a non-nucleotide linker.

The target capture reagent used contained the SARS-CoV capture probe at a concentration of 4 pmol/mL and the internal control capture probe at a concentration of 4.4 pmol/mL. From this stock, 400 μL of the target capture reagent containing approximately 250 copies of the internal control was added to each tube of the TTUs used for this experiment. Samples containing virus (HIV, HCV and HBV were inactivated) were provided to the tubes in 500 μL aliquots as set forth in Table 1 below. There were 10 replicates assayed for each concentration of the samples indicated, except for the HCoV-229E sample, for which there were only 5 replicates assayed. There were also 10 negative controls assayed containing an internal control only.

TABLE 1
Sample Concentrations

| Sample | Buffer | Titer | |
|---|---|---|---|
| HCoV-229E | Serum | 355 | TCID₅₀/mL |
| HIV | Serum | 500 | c/mL |
| HCV | Serum | 700 | c/mL |
| HBV | Serum | 100 | IU/mL |
| Parvovirus | Serum | 8000 | IU/mL |
| SARS Transcript | Target Capture Reagent | 1.25-400 | c/mL |

The HCoV-229E was obtained from the American Type Culture Collection in Manassass, Va. as ATCC number VR-740. Under the “Titer” heading in Table 1, “TCID₅₀” stands for 50% tissue culture infectious dose, “c” stands for copies, and “IU” stands for international units.

In the final step of the target capture protocol, all residual wash buffer was removed from the tubes. Following the target capture step, 75 μL of the primer/promoter-primer containing amplification reagent of Example 1 was added to each tube. As in Example 1, the final concentration of the primers and promoter-primers was 150 pmol/mL each per tube. The tubes were provided with 200 μL of a silicone oil, covered and vortexed for 10 to 20 seconds before incubating the TTUs in a 60° C. water bath for 10 minutes. The TTUs were then incubated in a 41.5° C. water bath for 10 minutes before adding 25 μL of the enzyme reagent of Example 1 to the tubes. After addition of the enzyme reagent, the TTUs were covered, removed from the water bath and hand shaken to fully mix the amplification and enzyme reagents. The TTUs were again placed in the 41.5° C. water bath and incubated for 60 minutes to facilitate amplification of the target sequences. Following amplification, the TTUs were removed from the 41.5° C. water bath and allowed to cool to room temperature.

For detection, 100 μL of a probe reagent (75 mM succinic acid, 3.5% (w/v) LLS, 75 mM LiOH, 15 mM aldrithiol-2, 1 M LiCl, 1 mM EDTA, 3% (v/v) ethyl alcohol, and LiOH to pH 4.2) spiked with the SARS-CoV detection probe to a concentration of 2.5×10⁷ RLU/mL and the internal control probe to a concentration of 7.5×10⁶ RLU/mL was added to each tube, where “RLU” stands for relative light units, a measure of chemiluminescence. After adding probe reagent, the TTUs were incubated in a 60° C. water bath for 15 minutes to permit hybridization of the detection probes to their corresponding target sequences contained in any amplification products of the SARS-CoV transcript or the internal control. Following hybridization, 250 μL of a selection reagent (600 mM boric acid, 235 mM NaOH, 1% (v/v) TRITON® X-100 detergent, and NaOH to pH 9) was added to the tubes, the TTUs were covered and vortexed for 10 to 20 seconds, and then incubated incubated in a 60° C. water bath for 10 minutes to hydrolyze acridinium ester labels associated with unhybridized probe. The samples were cooled in a water bath held at 19° to 27° C. for about 10 minutes before being analyzed in a LEADER® HC+ Luminometer (Gen-Probe; Cat. No. 4747) equipped with automatic injection of a solution containing 1 mM nitric acid and 0.1% (v/v) hydrogen peroxide, followed by automatic injection of a solution containing 1 N sodium hydroxide.

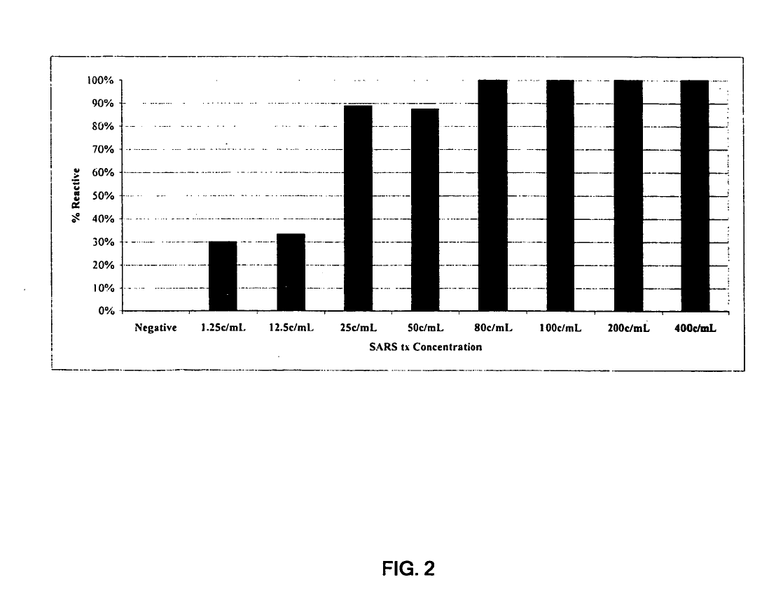

The results are summarized in Table 2 below and indicate that the SARS-CoV assay was 100% reactive at 80 c/mL and nearly 90% reactive at both 25 and 50 c/mL, as graphically represented in FIG. 2. The results of this assay further indicate that the SARS-CoV detection probe did not cross-react with HCoV-229E, HIV, HBV, parvovirus or 9 of the 10 HCV replicates. However, it is believed that the one reactive HCV replicate was the result of a cross-contaminated sample, as a BLAST search did not indicate any sequence similarity between the SARS-CoV detection probe and HCV RNA.

TABLE 2
Sensitivity and Specificity of SARS-CoV Assay

| | | | Flasher | | Glower | | |
|---|---|---|---|---|---|---|---|
| Source | Conc. | | Avg. | % CV | Avg. | % CV | % Reactivity |
| SARS-CoV | Negative | | 179376 | 25 | 682 | 105 | 0 |
| | 1.25 | c/mL | 211832 | 23 | 104898 | 218 | 30 |
| | 12.5 | c/mL | 217492 | 15 | 126823 | 195 | 33 |
| | 25 | c/mL | 235373 | 29 | 423433 | 78 | 89 |
| | 50 | c/mL | 242577 | 37 | 549103 | 45 | 88 |
| | 80 | c/mL | 262386 | 26 | 673600 | 30 | 100 |
| | 100 | c/mL | 227627 | 43 | 697568 | 24 | 100 |
| | 200 | c/mL | 299985 | 3 | 769405 | 1 | 100 |
| | 400 | c/mL | 293989 | 6 | 768587 | 1 | 100 |
| HCoV-229E | 355 | TCID₅₀/mL | 147326 | 10 | 220 | 139 | 0 |
| HIV | 500 | c/mL | 141582 | 25 | 1909 | 174 | 0 |
| HCV | 700 | c/mL | 160297 | 41 | 864 | 117 | 0 |
| HBV | 100 | IU/mL | 178992 | 13 | 2145 | 115 | 0 |
| Parvovirus | 8000 | IU/mL | 194746 | 10 | 606 | 136 | 0 |

The coefficient of variation values (“% CV”) appearing in Table 2 for the different copy levels tested constitute the standard deviation of the replicates over the mean of the replicates as a percentage. These values are generally larger with decreasing concentration of the transcript because some of the replicates were amplified, while others were not, thereby resulting in a higher standard deviation between the replicates.

We note that in other experiments incorporating an internal control derived from HIV nucleic acid, it was believed that certain primers were cross-hybridizing with the internal control or its primers. These primers included a primer having a base sequence perfectly complementary to that of SEQ ID NO:33 and primers having the following sequences:

| | |
|---|---|
| caagtcaatggttaccctaatatg, | SEQ ID NO:47 |
| ctaatatgtttatcacccgcg, | SEQ ID NO:48 |
| and | |
| caatggttaccctaatatgtttat. | SEQ ID NO:49 |

For that reason, the non-T7 primers of SEQ ID NO:42 and SEQ ID NO:43 were preferred in the above examples. Non-T7 primers targeting the base sequences of SEQ ID NO:28, SEQ ID NO:30, SEQ ID NO:32 and SEQ ID NO:33 could still be used to amplify SARS-CoV nucleic acid, however, attention would have to be given to the selection of an internal control and associated primers, if an internal control is to be included in an assay.

While the present invention has been described and shown in considerable detail with reference to certain preferred embodiments, those skilled in the art will readily appreciate other embodiments of the present invention. Accordingly, the present invention is deemed to include all modifications and variations encompassed within the spirit and scope of the following appended claims.

What is claimed is:

1. A detection probe for use determining the presence of SARS-CoV in a test sample, said probe being up to 100 bases in length and comprising a target binding portion which forms a hybrid stable for detection with a target sequence contained within the sequence of SEQ ID NO:1 or its complement under stringent hybridization conditions, wherein said probe does not form a hybrid stable for detection with nucleic acid derived from HCoV-OC43 or HCoV-229E under said conditions.

2. The probe of claim 1, wherein said target binding portion comprises an at least 10 contiguous base region which is perfectly complementary to an at least 10 contiguous base region of said target sequence or its complement.

3. The probe of claim 1, wherein said target binding portion comprises an at least 15 contiguous base region which is perfectly complementary to an at least 15 contiguous base region of said target sequence or its complement.

4. The probe of claim 1, wherein said probe comprises a base sequence selected from the group consisting of SEQ ID NO:2, its complement, and the RNA equivalents thereof.

5. The probe of claim 1, wherein the base sequence of said probe consists of a base sequence selected from the group consisting of SEQ ID NO:2, its complement, and the RNA equivalents thereof.

6. The probe of claim 1, wherein the base sequence of said target binding portion is perfectly complementary to all or a portion of the base sequence of SEQ ID NO:1 or its complement, and wherein said probe does not comprise any other base sequences which stably hybridize to nucleic acid derived from SARS-CoV under said conditions.

7. The probe of claim 6, wherein said target binding portion comprises an at least 10 contiguous base region which is perfectly complementary to an at least 10 contiguous base region of SEQ ID NO:1 or its complement.

8. The probe of claim 6, wherein said target binding portion comprises an at least 15 contiguous base region which is perfectly complementary to an at least 15 contiguous base region of SEQ ID NO:1 or its complement.

9. The probe of claim 6, wherein said probe is a self-hybridizing probe under said conditions and in the absence of said target sequence.

10. The probe of claim 9, wherein said probe comprises a pair of interacting labels.

11. The probe of claim 10, wherein said pair of interacting labels is selected from the group consisting of a luminescent/quencher pair, a luminescent/adduct pair, a Förrester energy transfer pair and a dye dimer.

12. The probe of claim 1, wherein said probe comprises a detectable label.

13. The probe of claim 1, wherein said conditions include a temperature of about 60° C. and a salt concentration of about 0.6 M to about 0.9 M.

14. A method for determining the presence of SARS-CoV in a test sample, said method comprising the steps of:
a) contacting a test sample with said probe of claim 1 under said conditions; and
b) determining whether said hybrid is present in said test sample as an indication of the presence of SARS-CoV in said test sample.

15. A detection probe for use determining the presence of SARS-CoV in a test sample, said probe being up to 100 bases in length and comprising a target binding portion which forms a hybrid stable for detection with a target sequence contained within the sequence of SEQ ID NO:3 or its complement under stringent hybridization conditions, wherein said probe does not form a hybrid stable for detection with nucleic acid derived from HCoV-OC43 or HCoV-229E under said conditions.

16. The probe of claim 15, wherein said target binding portion comprises an at least 10 contiguous base region which is perfectly complementary to an at least 10 contiguous base region of said target sequence or its complement.

17. The probe of claim 15, wherein said target binding portion comprises an at least 15 contiguous base region which is perfectly complementary to an at least 15 contiguous base region of said target sequence or its complement.

18. The probe of claim 15, wherein the base sequence of said target binding portion is perfectly complementary to all or a portion of the base sequence of SEQ ID NO:3 or its complement, and wherein said probe does not comprise any other base sequences which stably hybridize to nucleic acid derived from SARS-CoV under said conditions.

19. The probe of claim 18, wherein said target binding portion comprises an at least 10 contiguous base region which is perfectly complementary to an at least 10 contiguous base region of SEQ ID NO:3 or its complement.

20. The probe of claim 18, wherein said target binding portion comprises an at least 15 contiguous base region which is perfectly complementary to an at least 15 contiguous base region of SEQ ID NO:3 or its complement.

21. The probe of claim 15, wherein said probe comprises a base sequence selected from the group consisting of SEQ ID NO:4, SEQ ID NO:5 and SEQ ID NO:6, their complements, and the RNA equivalents thereof.

22. The probe of claim 15, wherein the base sequence of said target binding portion is contained within a base sequence selected from the group consisting of SEQ ID NO:4, SEQ ID NO:5 and SEQ ID NO:6, their complements, and the RNA equivalents thereof, and wherein said probe does not comprise any other base sequences which stably hybridize to nucleic acid derived from SARS-CoV under said conditions.

23. The probe of claim 22, wherein the base sequence of said target binding portion is contained within a base sequence selected from the group consisting of SEQ ID NO:4, its complement, and the RNA equivalents thereof.

24. The probe of claim 23, wherein the base sequence of said target binding portion consists of a base sequence selected from the group consisting of SEQ ID NO:4, its complement, and the RNA equivalents thereof.

25. The probe of claim 24, wherein the base sequence of said probe consists of a base sequence selected from the group consisting of SEQ ID NO:4, its complement, and the RNA equivalents thereof.

26. The probe of claim 22, wherein the base sequence of said target binding portion is contained within a base sequence selected from the group consisting of SEQ ID NO:5, its complement, and the RNA equivalents thereof.

27. The probe of claim 26, wherein the base sequence of said target binding portion consists of a base sequence selected from the group consisting of SEQ ID NO:5, its complement, and the RNA equivalents thereof.

28. The probe of claim 27, wherein the base sequence of said probe consists of a base sequence selected from the group consisting of SEQ ID NO:5, its complement, and the RNA equivalents thereof.

29. The probe of claim 22, wherein the base sequence of said target binding portion is contained within a base sequence selected from the group consisting of SEQ ID NO:6, its complement, and the RNA equivalents thereof.

30. The probe of claim 29, wherein the base sequence of said target binding portion consists of a base sequence selected from the group consisting of SEQ ID NO:6, its complement, and the RNA equivalents thereof.

31. The probe of claim 30, wherein the base sequence of said probe consists of a base sequence selected from the group consisting of SEQ ID NO:6, its complement, and the RNA equivalents thereof.

32. The probe of claim 18, wherein said probe is a self-hybridizing probe under said conditions and in the absence of said target sequence.

33. The probe of claim 32, wherein said probe comprises a pair of interacting labels.

34. The probe of claim 33, wherein said pair of interacting labels is selected from the group consisting of a luminescent/quencher pair, a luminescent/adduct pair, a Förrester energy transfer pair and a dye dimer.

35. The probe of claim 15, wherein said probe comprises a detectable label.

36. The probe of claim 15, wherein said conditions include a temperature of about 60° C. and a salt concentration of about 0.6 M to about 0.9 M.

37. A method for determining the presence of SARS-CoV in a test sample, said method comprising the steps of:
a) contacting a test sample with said probe of claim 16 under said conditions; and
b) determining whether said hybrid is present in said test sample as indication of the presence of SARS-CoV in said test sample.

38. An oligonucleotide set comprising two or more oligonucleotides capable of amplifying a target region of nucleic acid derived from SARS-CoV under amplification conditions, said target region being contained within the sequence of SEQ ID NO:8 or its complement.

39. The oligonucleotide set of claim 38, wherein said set comprises:
a first oligonucleotide up to 100 bases in length which binds to or extends through a first target sequence contained within the sequence of SEQ ID NO:9 or its complement under amplification conditions; and
a second oligonucleotide up to 100 bases in length which binds to or extends through a second target sequence contained within the sequence of SEQ ID NO:10 or its complement under amplification conditions.

40. The oligonucleotide set of claim 39, wherein said first target sequence is selected from the group consisting of SEQ ID NO:11, SEQ ID NO:12, SEQ ID NO:13, SEQ ID NO:14, SEQ ID NO:15, SEQ ID NO:16, SEQ ID NO:17, SEQ ID NO:18 and SEQ ID NO:19, and their complements.

41. The oligonucleotide set of claim 39, wherein said second target sequence is selected from the group consisting of SEQ ID NO:20, SEQ ID NO:21 and SEQ ID NO:22, and their complements.

42. The oligonucleotide set of claim 40, wherein said second target sequence is selected from the group consisting of SEQ ID NO:20, SEQ ID NO:21 and SEQ ID NO:22, and their complements.

43. The oligonucleotide set of claim 39, wherein said first oligonucleotide comprises a base sequence selected from the group consisting of SEQ ID NO:11, SEQ ID NO:12, SEQ ID NO:13, SEQ ID NO:14, SEQ ID NO:15, SEQ ID NO:16, SEQ ID NO:17, SEQ ID NO:18 and SEQ ID NO:19, their complements, and the DNA equivalents thereof.

44. The oligonucleotide set of claim 39, wherein said second oligonucleotide comprises a base sequence selected from the group consisting of SEQ ID NO:20, SEQ ID NO:21 and SEQ ID NO:22, their complements, and the DNA equivalents thereof.

45. The oligonucleotide set of claim 43, wherein said second oligonucleotide comprises a base sequence selected from the group consisting of SEQ ID NO:20, SEQ ID NO:21 and SEQ ID NO:22, their complements, and the DNA equivalents thereof.

46. The oligonucleotide set of claim 39, wherein the base sequence of said first oligonucleotide is contained within a base sequence selected from the group consisting of SEQ ID NO:11, SEQ ID NO:12, SEQ ID NO:13, SEQ ID NO:14, SEQ ID NO:15, SEQ ID NO:16, SEQ ID NO:17, SEQ ID NO:18 and SEQ ID NO:19, their complements, the DNA equivalents thereof, and any of the foregoing in combination with a 5′ sequence which is recognized by an RNA polymerase or which enhances initiation or elongation by RNA polymerase.

47. The oligonucleotide set of claim 39, wherein the base sequence of said second oligonucleotide is contained within a base sequence selected from the group consisting of SEQ ID NO:20, SEQ ID NO:21 and SEQ ID NO:22, their complements, the DNA equivalents thereof, and any of the foregoing in combination with a 5′ sequence which is recognized by an RNA polymerase or which enhances initiation or elongation by RNA polymerase.

48. The oligonucleotide set of claim 46, wherein the base sequence of said second oligonucleotide is contained within a base sequence selected from the group consisting of SEQ ID NO:20, SEQ ID NO:21 and SEQ ID NO:22, their complements, the DNA equivalents thereof, and any of the foregoing in combination with a 5′ sequence which is recognized by an RNA polymerase or which enhances initiation or elongation by RNA polymerase.

49. The oligonucleotide set of claim 39, wherein the base sequence of said first oligonucleotide consists of a base sequence selected from the group consisting of SEQ ID NO:11, SEQ ID NO:12, SEQ ID NO:13, SEQ ID NO:14, SEQ ID NO:15, SEQ ID NO:16, SEQ ID NO:17, SEQ ID NO:18 and SEQ ID NO:19, their complements, the DNA equivalents thereof, and any of the foregoing in combination with a 5′ sequence which is recognized by an RNA polymerase or which enhances initiation or elongation by RNA polymerase.

50. The oligonucleotide set of claim 39, wherein the base sequence of said second oligonucleotide consists of a base sequence selected from the group consisting of SEQ ID NO:20, SEQ ID NO:21 and SEQ ID NO:22, their complements, the DNA equivalents thereof, and any of the foregoing in combination with a 5′ sequence which is recognized by an RNA polymerase or which enhances initiation or elongation by RNA polymerase.

51. The oligonucleotide set of claim 49, wherein the base sequence of said second oligonucleotide consists of a base sequence selected from the group consisting of SEQ ID NO:20, SEQ ID NO:21 and SEQ ID NO:22, their complements, the DNA equivalents thereof, and any of the foregoing in combination with a 5′ sequence which is recognized by an RNA polymerase or which enhances initiation or elongation by RNA polymerase.

52. The oligonucleotide set of claim 51, wherein at least one of said first and second oligonucleotides comprises a T7 promoter sequence.

53. The oligonucleotide set of claim 38 further comprising a third oligonucleotide for use in determining the presence of a target sequence derived from said target region of SARS-CoV-derived nucleic acid, said third oligonucleotide being up to 100 bases in length and comprising a target binding portion which forms a hybrid stable for detection with said target sequence under stringent hybridization conditions, wherein said third oligonucleotide does not form a hybrid stable for detection with nucleic acid derived from HCoV-OC43 or HCoV-229E under said stringent hybridization conditions.

54. The oligonucleotide set of claim 53, wherein said target binding portion is complementary to the sequence of SEQ ID NO:1 or its complement.

55. The oligonucleotide set of claim 54, wherein the base sequence of said target binding portion is contained within a base sequence selected from the group consisting of SEQ ID NO:1, its complement, and the DNA equivalents thereof, and wherein said third oligonucleotide does not comprise any other base sequences which stably hybridize to nucleic acid derived from SARS-CoV under said stringent hybridization conditions.

56. The oligonucleotide set of claim 55, wherein said third oligonucleotide is a self-hybridizing oligonucleotide under said stringent hybridization conditions and in the absence of said target sequence.

57. The oligonucleotide of claim 56, wherein said third oligonucleotide comprises a pair of interacting labels.

58. The oligonucleotide set of claim 53, wherein the base sequence of said third oligonucleotide consists of a base sequence selected from the group consisting of SEQ ID NO:2, its complement, and the RNA equivalents thereof.

59. The oligonucleotide set of claim 53, wherein said third oligonucleotide comprises a detectable label.

60. The oligonucleotide set of claim 38 further comprising a third oligonucleotide for use in isolating and purifying a target nucleic acid containing said target region of SARS-CoV-derived nucleic acid, said third oligonucleotide being up to 100 bases in length and comprising a target binding portion that is complementary to a target sequence selected from the group consisting of SEQ ID NO:35, SEQ ID NO:36 and SEQ ID NO:37, wherein said third oligonucleotide stably hybridizes to said target sequence under assay conditions.

61. The oligonucleotide set of claim 60, wherein the base sequence of said target binding portion of said third oligonucleotide is contained within a base sequence selected from the group consisting of the complements of SEQ ID NO:35, SEQ ID NO:36 and SEQ ID NO:37, and the DNA equivalents thereof, and wherein said third oligonucleotide does not comprise any other base sequences which stably hybridize to SARS-CoV-derived nucleic acid under said assay conditions.

62. The oligonucleotide set of claim 61, wherein the base sequence of said target binding portion of said third oligonucleotide consists of a base sequence selected from the group consisting of the complements of SEQ ID NO:35, SEQ ID NO:36 and SEQ ID NO:37, and the DNA equivalents thereof.

63. The oligonucleotide set of claim 53 further comprising a fourth oligonucleotide for use in isolating and purifying a target nucleic acid containing said target region of SARS-CoV-derived nucleic acid, said fourth oligonucleotide being up to 100 bases in length and comprising a target binding portion that is complementary to a target sequence selected from the group consisting of SEQ ID NO:35, SEQ ID NO:36 and SEQ ID NO:37, wherein said fourth oligonucleotide stably hybridizes to said target sequence under assay conditions.

64. A method for amplifying a target region of nucleic acid derived from SARS-CoV, said method comprising the steps of:
a) contacting a test sample with said two or more oligonucleotides of claim 38; and
b) exposing said test sample to said amplification conditions such that said target region, if present in said test sample, is amplified.

65. A method for determining the presence of SARS-CoV in a test sample, said method comprising the steps of:
a) contacting a test sample with said first and second oligonucleotides of claim 39 under said amplification conditions;
b) amplifying, if present in said test sample, said target region;
c) contacting said test sample a third oligonucleotide, said third oligonucleotide being up to 100 bases in length and comprising a target binding portion which forms a hybrid stable for detection with a sequence contained within or complementary to said target region under stringent hybridization conditions, wherein said third oligonucleotide does not form a hybrid stable for detection with nucleic acid derived from HCoV-OC43 or HCoV-229E under said stringent hybridization conditions; and
d) determining whether said hybrid is present in said test sample as indication of the presence of SARS-CoV in said test sample.

66. The method of claim 65, wherein said third oligonucleotide is provided to said test sample prior to or during said amplifying step.

67. The method of claim 66, wherein at least a portion of said determining step occurs during said amplifying step.

68. An oligonucleotide set comprising two or more oligonucleotides capable of amplifying a target region of nucleic acid derived from SARS-CoV under amplification conditions, said target region being contained within the sequence of SEQ ID NO:23 or its complement.

69. The oligonucleotide set of claim 68, wherein said set comprises:
a first oligonucleotide up to 100 bases in length which binds to or extends through a first target sequence contained within the sequence of SEQ ID NO:24 or its complement under amplification conditions; and
a second oligonucleotide up to 100 bases in length which binds to or extends through a second target sequence contained within the sequence of SEQ ID NO:25 or its complement under amplification conditions.

70. The oligonucleotide set of claim 69, wherein said first target sequence is selected from the group consisting of SEQ ID NO:26 and SEQ ID NO:27, and their complements.

71. The oligonucleotide set of claim 69, wherein said second target sequence is selected from the group consisting of SEQ ID NO:28, SEQ ID NO:29, SEQ ID NO:30, SEQ ID NO:31, SEQ ID NO:32 and SEQ ID NO:33, and their complements.

72. The oligonucleotide set of claim 70, wherein said second target sequence is selected from the group consisting of SEQ ID NO:28, SEQ ID NO:29, SEQ ID NO:30, SEQ ID NO:31, SEQ ID NO:32 and SEQ ID NO:33, and their complements.

73. The oligonucleotide set of claim 69, wherein said first oligonucleotide comprises a base sequence selected from the group consisting of SEQ ID NO:26 and SEQ ID NO:27, their complements, and the DNA equivalents thereof.

74. The oligonucleotide set of claim 69, wherein said second oligonucleotide comprises a base sequence selected from the group consisting of SEQ ID NO:28, SEQ ID NO:29, SEQ ID NO:30, SEQ ID NO:31, SEQ ID NO:32 and SEQ ID NO:33, their complements, and the DNA equivalents thereof.

75. The oligonucleotide set of claim 73, wherein said second oligonucleotide comprises a base sequence selected from the group consisting of SEQ ID NO:28, SEQ ID NO:29, SEQ ID NO:30, SEQ ID NO:31, SEQ ID NO:32 and SEQ ID NO:33, their complements, and the DNA equivalents thereof.

76. The oligonucleotide set of claim 69, wherein the base sequence of said first oligonucleotide is contained within a base sequence selected from the group consisting of SEQ ID NO:26 and SEQ ID NO:27, their complements, the DNA equivalents thereof, and any of the foregoing in combination with a 5′ sequence which is recognized by an RNA polymerase or which enhances initiation or elongation by RNA polymerase.

77. The oligonucleotide set of claim 69, wherein the base sequence of said second oligonucleotide is contained within a base sequence selected from the group consisting of SEQ ID NO:28, SEQ ID NO:29, SEQ ID NO:30, SEQ ID NO:31, SEQ ID NO:32 and SEQ ID NO:33, their complements, the DNA equivalents thereof, and any of the foregoing in combination with a 5′ sequence which is recognized by an RNA polymerase or which enhances initiation or elongation by RNA polymerase.

78. The oligonucleotide set of claim 76, wherein the base sequence of said second oligonucleotide is contained within a base sequence selected from the group consisting of SEQ ID NO:28, SEQ ID NO:29, SEQ ID NO:30, SEQ ID NO:31, SEQ ID NO:32 and SEQ ID NO:33, their complements, the DNA equivalents thereof, and any of the foregoing in combination with a 5′ sequence which is recognized by an RNA polymerase or which enhances initiation or elongation by RNA polymerase.

79. The oligonucleotide set of claim 69, wherein the base sequence of said first oligonucleotide consists of a base sequence selected from the group consisting of SEQ ID NO:26 and SEQ ID NO:27, their complements, the DNA equivalents thereof, and any of the foregoing in combination with a 5′ sequence which is recognized by an RNA polymerase or which enhances initiation or elongation by RNA polymerase.

80. The oligonucleotide set of claim 69, wherein the base sequence of said second oligonucleotide consists of a base sequence selected from the group consisting of SEQ ID NO:28, SEQ ID NO:29, SEQ ID NO:30, SEQ ID NO:31, SEQ ID NO:32 and SEQ ID NO:33, their complements, the DNA equivalents thereof, and any of the foregoing in combination with a 5′ sequence which is recognized by an RNA polymerase or which enhances initiation or elongation by RNA polymerase.

81. The oligonucleotide set of claim 79, wherein the base sequence of said second oligonucleotide consists of a base sequence selected from the group consisting of SEQ ID NO:28, SEQ ID NO:29, SEQ ID NO:30, SEQ ID NO:31, SEQ ID NO:32 and SEQ ID NO:33, their complements, the DNA equivalents thereof, and any of the foregoing in combination with a 5′ sequence which is recognized by an RNA polymerase or which enhances initiation or elongation by RNA polymerase.

82. The oligonucleotide set of claim 81, wherein at least one of said first and second oligonucleotides comprises a T7 promoter sequence.

83. The oligonucleotide set of claim 68 further comprising a third oligonucleotide for use in determining the presence of a target sequence derived from said target region of SARS-CoV-derived nucleic acid, said third oligonucleotide being up to 100 bases in length and comprising a target binding portion which forms a hybrid stable for detection with said target sequence under stringent hybridization conditions, wherein said third oligonucleotide does not form a hybrid stable for detection with nucleic acid derived from HCoV-OC43 or HCoV-229E under said stringent hybridization conditions.

84. The oligonucleotide set of claim 83, wherein said target binding portion is complementary to the sequence of SEQ ID NO:3 or its complement.

85. The oligonucleotide set of claim 83, wherein the base sequence of said target binding portion is contained within a base sequence selected from the group consisting of SEQ ID NO:3, its complement, and the DNA equivalents thereof, and wherein said third oligonucleotide does not comprise any other base sequences which stably hybridize to nucleic acid derived from SARS-CoV under said stringent hybridization conditions.

86. The oligonucleotide set of claim 85, wherein the base sequence of said target binding portion is contained within a base sequence selected from the group consisting of SEQ ID NO:4, SEQ ID NO:5 and SEQ ID NO:6, their complements, and the RNA equivalents thereof, and wherein said third oligonucleotide does not comprise any other base sequences which stably hybridize to nucleic acid derived from SARS-CoV under said stringent hybridization conditions.

87. The oligonucleotide set of claim 86, wherein said third oligonucleotide is a self-hybridizing oligonucleotide under said stringent hybridization conditions and in the absence of said target sequence.

88. The oligonucleotide of claim 87, wherein said third oligonucleotide comprises a pair of interacting labels.

89. The oligonucleotide set of claim 83, wherein the base sequence of said third oligonucleotide consists of a base sequence selected from the group consisting of SEQ ID NO:4, SEQ ID NO:5 and SEQ ID NO:6, their complements, and the RNA equivalents thereof.

90. The oligonucleotide set of claim 83, wherein said third oligonucleotide comprises a detectable label.

91. The oligonucleotide set of claim 68 further comprising a third oligonucleotide for use in isolating and purifying a target nucleic acid containing said target region of SARS-CoV-derived nucleic acid, said third oligonucleotide being up to 100 bases in length and comprising a target binding portion that is complementary to a target sequence selected from the group consisting of SEQ ID NO:35, SEQ ID NO:36 and SEQ ID NO:37, and wherein said third oligonucleotide stably hybridizes to said target sequence under assay conditions.

92. The oligonucleotide set of claim 91, wherein the base sequence of said target binding portion of said third oligonucleotide is contained within a base sequence selected from the group consisting of the complements of SEQ ID NO:35, SEQ ID NO:36 and SEQ ID NO:37, and the DNA equivalents thereof, and wherein said third oligonucleotide does not comprise any other base sequences which stably hybridize to SARS-CoV-derived nucleic acid under said assay conditions.

93. The oligonucleotide set of claim 92, wherein the base sequence of said target binding portion of said third oligonucleotide consists of a base sequence selected from the group consisting of the complements of SEQ ID NO:35, SEQ ID NO:36 and SEQ ID NO:37 and the DNA equivalents thereof.

94. The oligonucleotide set of claim 83 further comprising a fourth oligonucleotide for use in isolating and purifying a target nucleic acid containing said target region of SARS-CoV-derived nucleic acid, said fourth oligonucleotide being up to 100 bases in length and comprising a target binding portion that is complementary to a target sequence selected from the group consisting of SEQ ID NO:35, SEQ ID NO:36 and SEQ ID NO:37, and wherein said fourth oligonucleotide stably hybridizes to said target sequence under assay conditions.

95. A method for amplifying a target region of nucleic acid derived from SARS-CoV, said method comprising the steps of:
a) contacting a test sample with said two or more oligonucleotides of claim 68; and
b) exposing said test sample to said amplification conditions such that said target region, if present in said test sample, is amplified.

96. A method for determining the presence of SARS-CoV in a test sample, said method comprising the steps of:
a) contacting a test sample with said first and second oligonucleotides of claim 69 under said amplification conditions;
b) amplifying, if present in said test sample, said target region;
c) contacting to said test sample a third oligonucleotide, said third oligonucleotide being up to 100 bases in length and comprising a target binding portion which forms a hybrid stable for detection with a sequence contained within or complementary to said target region under stringent hybridization conditions, wherein said third oligonucleotide does not form a hybrid stable for detection with nucleic acid derived from HCoV-OC43 or HCoV-229E under said stringent hybridization conditions; and
d) determining whether said hybrid is present in said test sample as indication of the presence of SARS-CoV in said test sample.

97. The method of claim 96, wherein said third oligonucleotide is provided to said test sample prior to or during said amplifying step.

98. The method of claim 97, wherein at least a portion of said determining step occurs during said amplifying step.

99. A method for determining the presence of SARS-CoV in a test sample, said method comprising the steps of:
a) contacting a test sample a detection probe up to 100 bases in length and comprising a target binding portion which forms a hybrid stable for detection with a target sequence contained within a SARS-CoV 5′ leader sequence or its complement, wherein said probe does not form a hybrid stable for detection with nucleic acid derived from HCoV-OC43 or HCoV-229E under said conditions; and
b) determining whether said hybrid is present in said test sample as an indication of the presence of SARS-CoV in said test sample.

100. The method of claim 99, wherein said target sequence comprises a core sequence of a transcription regulating sequence or its complement.

101. The method of claim 100, wherein said core sequence consists of at least 5 contiguous nucleotides of the sequence of SEQ ID NO:38 or its complement.

102. The method of claim 101, wherein the core sequence consists of the sequence of SEQ ID NO:38 or its complement.

103. The method of claim 99 further comprising contacting said test sample with a pair of amplification oligonucleotides under amplification conditions, wherein each member of said pair of amplification oligonucleotides comprises a target binding portion which binds to or extends through at least a portion of said 5′ leader sequence or its complement under said amplification conditions.

104. The method of claim 103, wherein said target binding portion of each member of said pair of amplification oligonucleotides binds to a target region fully contained within said 5′ leader sequence or its complement under said amplification conditions, and wherein said amplification oligonucleotides do not contain any other base sequences which stably hybridize to nucleic acid derived from SARS-CoV under said amplification conditions.

105. The method of claim 99 further comprising contacting said test sample with a pair of amplification oligonucleotides under amplification conditions, wherein a target binding portion of a first member of said pair of amplification oligonucleotides binds to a target region fully contained within said 5′ leader sequence or its complement under said amplification conditions, and wherein a target binding portion of a second member of said pair of amplification oligonucleotides binds to a target region fully contained within a SARS-CoV 3′ co-terminal sequence or its complement under said amplification conditions, wherein said amplification oligonucleotides do not contain any other base sequences which stably hybridize to nucleic acid derived from SARS-CoV under said amplification conditions.

106. A method for amplifying a target region of nucleic acid derived from SARS-CoV, said method comprising the steps of:
a) contacting a test sample with one or more amplification oligonucleotides under amplification conditions, wherein a first of said amplification oligonucleotides comprises a target binding portion which binds to a target region fully contained within a SARS-CoV 5′ leader sequence or its complement under said conditions, wherein said first amplification oligonucleotide does not contain any other base sequences which stably hybridize to nucleic acid derived from SARS-CoV under said amplification conditions; and
b) exposing said test sample to said conditions such that said target region, if present in said test sample, is amplified.

107. The method of claim 106, wherein said target region comprises a core sequence of a transcription regulating sequence or its complement.

108. The method of claim 107, wherein said core sequence consists of at least 5 contiguous nucleotides of the sequence of SEQ ID NO:38 or its complement.

109. The method of claim 108, wherein the core sequence consists of the sequence of SEQ ID NO:38 or its complement.

110. The method of claim 106, wherein said test sample is contacted with a pair of said amplification oligonucleotides under said conditions, wherein a second of said amplification oligonucleotides comprises a target binding portion which binds to a target region fully contained within said 5′ leader sequence or its complement under said conditions, and wherein said second amplification oligonucleotide does not contain any other base sequences which stably hybridize to nucleic acid derived from SARS-CoV under said conditions.

111. A method for determining the presence of SARS-CoV in a test sample, said method comprising the steps of:
a) contacting a test sample with a detection probe up to 100 bases in length and comprising a target binding portion which forms a hybrid stable for detection with a target sequence contained within a SARS-CoV 3′ co-terminal sequence or its complement, wherein said probe forms a hybrid stable for detection with said target sequence under stringent hybridization conditions, and wherein said probe does not form a hybrid stable for detection with nucleic acid derived from HCoV-OC43 or HCoV-229E under said conditions; and
b) determining whether said hybrid is present in said test sample as an indication of the presence of SARS-CoV in said test sample.

112. The method of claim 111 further comprising contacting said test sample with a pair of amplification oligonucleotides under amplification conditions, wherein each member of said pair of amplification oligonucleotides comprises a target binding portion which binds or extends through at least a portion of said 3′ co-terminal sequence or its complement under said amplification conditions.

113. The method of claim 111, wherein said target binding portion of each member of said pair of amplification oligonucleotides binds to a target region fully contained within said 3′ co-terminal sequence or its complement under said amplification conditions, and wherein said amplification oligonucleotides do not contain any other base sequences which stably hybridize to nucleic acid derived from SARS-CoV under said amplification conditions.

114. A method for amplifying a target region of nucleic acid derived from SARS-CoV, said method comprising the steps of:
a) contacting a test sample with one or more amplification oligonucleotides under amplification conditions, wherein a first of said amplification oligonucleotides comprises a target binding portion which binds to a target region contained within a SARS-CoV 3′ co-terminal sequence or its complement under said conditions; and
b) exposing said test sample to said conditions such that said target region, if present in said test sample, is amplified.

115. The method of claim 114, wherein said test sample is contacted with a pair of said amplification oligonucleotides under said conditions, wherein a second of said amplification oligonucleotides comprises a target binding portion which binds to a target region fully contained within said 3′ co-terminal sequence or its complement under said conditions, and wherein said second amplification oligonucleotide does not contain any other base sequences which stably hybridize to nucleic acid derived from SARS-CoV under said conditions.


SEQUENCE LISTING (49 sequences)

SEQ ID NO: 1 - RNA, 21 nt, SARS coronavirus
ccuuauggguugggauuaucc

SEQ ID NO: 2 - DNA, 21 bp, Artificial
  DNA equivalent of a SARS coronavirus sequence
ccttatgggttgggattatcc

SEQ ID NO: 3 - RNA, 23 nt, SARS coronavirus
cgugcguggauuggcuuugaugu

SEQ ID NO: 4 - DNA, 18 bp, Artificial
  DNA equivalent of a SARS coronavirus sequence
cgtgcgtggattggcttt

SEQ ID NO: 5 - DNA, 19 bp, Artificial
  DNA equivalent of a SARS coronavirus sequence
cgtgcgtggattggctttg

SEQ ID NO: 6 - DNA, 21 bp, Artificial
  DNA equivalent of a SARS coronavirus sequence
tgcgtggattggctttgatgt

SEQ ID NO: 7 - RNA, 24 nt, Artificial
  Hairpin molecule containing 2′-O-methyl equivalent of a SARS coronavirus sequence
ccgugcguggauuggcuuucacgg

SEQ ID NO: 8 - RNA, 182 nt, SARS coronavirus
cugugguaauuggaacaagcaaguuuuacgguggcuggcauaauauguuaaaaacuguuuacagugaugagaaacuccacaccuuauggguugggauuauccaaaaugugacagagccaugccuaacaugcuuaggauaauggccucucuuguucuugcucgcaaacauaacacuugcugua

SEQ ID NO: 9 - RNA, 85 nt, SARS coronavirus
uauccaaaaugugacagagccaugccuaacaugcuuaggauaauggccucucuuguucuugcucgcaaacauaacacuugcugua

SEQ ID NO: 10 - RNA, 38 nt, SARS coronavirus
cugugguaauuggaacaagcaaguuuuacgguggcugg

SEQ ID NO: 11 - RNA, 26 nt, SARS coronavirus
uauccaaaaugugacagagccaugcc

SEQ ID NO: 12 - RNA, 24 nt, SARS coronavirus
auccaaaaugugacagagccaugc

SEQ ID NO: 13 - RNA, 23 nt, SARS coronavirus
ccaaaaugugacagagccaugcc

SEQ ID NO: 14 - RNA, 23 nt, SARS coronavirus
aaaugugacagagccaugccuaa

SEQ ID NO: 15 - RNA, 26 nt, SARS coronavirus
ugugacagagccaugccuaacaugcu

SEQ ID NO: 16 - RNA, 25 nt, SARS coronavirus
gugacagagccaugccuaacaugcu

SEQ ID NO: 17 - RNA, 23 nt, SARS coronavirus
augccuaacaugcuuaggauaau

SEQ ID NO: 18 - RNA, 21 nt, SARS coronavirus
augcuuaggauaauggccucu

SEQ ID NO: 19 - RNA, 25 nt, SARS coronavirus
gcucgcaaacauaacacuugcugua

SEQ ID NO: 20 - RNA, 25 nt, SARS coronavirus
cugugguaauuggaacaagcaaguu

SEQ ID NO: 21 - RNA, 19 nt, SARS coronavirus
gaacaagcaaguuuuacgg

SEQ ID NO: 22 - RNA, 22 nt, SARS coronavirus
aagcaaguuuuacgguggcugg

SEQ ID NO: 23 - RNA, 112 nt, SARS coronavirus
caagucaaugguuacccuaauauguuuaucacccgcgaagaagcuauucgucacguucgugcguggauuggcuuugauguagagggcugucaugcaacuagagaugcugugg

SEQ ID NO: 24 - RNA, 31 nt, SARS coronavirus
gagggcugucaugcaacuagagaugcugugg

SEQ ID NO: 25 - RNA, 45 nt, SARS coronavirus
caagucaaugguuacccuaauauguuuaucacccgcgaagaagcu

SEQ ID NO: 26 - RNA, 21 nt, SARS coronavirus
gagggcugucaugcaacuaga

SEQ ID NO: 27 - RNA, 22 nt, SARS coronavirus
caugcaacuagagaugcugugg

SEQ ID NO: 28 - RNA, 24 nt, SARS coronavirus
caagucaaugguuacccuaauaug

SEQ ID NO: 29 - RNA, 23 nt, SARS coronavirus
gucaaugguuacccuaauauguu

SEQ ID NO: 30 - RNA, 24 nt, SARS coronavirus
caaugguuacccuaauauguuuau

SEQ ID NO: 31 - RNA, 22 nt, SARS coronavirus
uuacccuaauauguuuaucacc

SEQ ID NO: 32 - RNA, 21 nt, SARS coronavirus
cuaauauguuuaucacccgcg

SEQ ID NO: 33 - RNA, 20 nt, SARS coronavirus
uuaucacccgcgaagaagcu

SEQ ID NO: 34 - DNA, 27 bp, Artificial
  T7 RNA polymerase promoter sequence
aatttaatacgactcactatagggaga

SEQ ID NO: 35 - RNA, 23 nt, SARS coronavirus
agacaguuucaucagaaauuauu

SEQ ID NO: 36 - RNA, 23 nt, SARS coronavirus
auauguuaaaccagguggaacau

SEQ ID NO: 37 - RNA, 29 nt, SARS coronavirus
gguguuaacuuaguagcuguaccgacugg

SEQ ID NO: 38 - RNA, 10 nt, SARS coronavirus
uaaaacgaac

SEQ ID NO: 39 - DNA, 33 bp, Artificial
  Homopolymer tail with flexible linker for use with a capture probe
tttaaaaaaaaaaaaaaaaaaaaaaaaaaaaaa

SEQ ID NO: 40 - DNA, 21 bp, Artificial
  SARS coronavirus complementary sequence
tctagttgcatgacagccctc

SEQ ID NO: 41 - DNA, 22 bp, Artificial
  SARS coronavirus complementary sequence
ccacagcatctctagttgcatg

SEQ ID NO: 42 - DNA, 22 bp, Artificial
  DNA equivalent of a SARS coronavirus sequence
ttaccctaatatgtttatcacc

SEQ ID NO: 43 - DNA, 23 bp, Artificial
  DNA equivalent of a SARS coronavirus sequence
gtcaatggttaccctaatatgtt

SEQ ID NO: 44 - RNA, 23 nt, Artificial
  Hairpin molecule containing 2′-O-methyl equivalent of a SARS coronavirus sequence
ccgucgucacguucgugcgacgg

SEQ ID NO: 45 - RNA, 24 nt, Artificial
  Hairpin molecule containing 2-O-methyl equivalent of a SARS coronavirus sequence
ccgacugauguagagggcugucgg

SEQ ID NO: 46 - DNA, 21 bp, Artificial
  2′-O-methyl/DNA equivalent of a SARS coronavirus sequence
ugcguggauuggcuuugaugt

SEQ ID NO: 47 - DNA, 24 bp, Artificial
  DNA equivalent of a SARS coronavirus sequence
caagtcaatggttaccctaatatg

SEQ ID NO: 48 - DNA, 21 bp, Artificial
  DNA equivalent of a SARS coronavirus sequence
ctaatatgtttatcacccgcg

SEQ ID NO: 49 - DNA, 24 bp, Artificial
  DNA equivalent of a SARS coronavirus sequence
caatggttaccctaatatgtttat